(19) United States
(12) Patent Application Publication
(10) Pub. No.: US 2004/0002106 A1
(43) Pub. Date: Jan. 1, 2004

(54) RNA cleaving DNA enzymes and methods of making and using same

(76) Inventors: Dipankar Sen, Burnaby (CA); Anat R. Feldman, Burnaby (CA)

Correspondence Address: OYEN, WIGGS, GREEN & MUTALA, 480 - THE STATION, 601 WEST CORDOVA STREET, VANCOUVER, BC, V6B 1G1, CA

(21) Appl. No.: 10/438,340

(22) Filed: May 15, 2003

Related U.S. Application Data
(60) Provisional application No. 60/378,071, filed on May 16, 2002.

(51) Int. Cl. C12Q 1/68; C07H 21/04; C12N 9/22

(52) U.S. Cl. 435/6; 435/199; 536/23.2

(57) ABSTRACT

The present invention discloses enzymatic DNA molecules capable of cleaving RNA, referred to herein as the “Bipartite DNAzymes.” The Bipartite I DNAzyme is capable of self-cleavage at an internal ribonucleotide. The Bipartite II DNAzyme is capable of sequence-specific cleavage of RNA substrates. The sequence of the substrate binding arms of the Bipartite II DNAzyme can be modified to allow the DNAzyme to bind to and to cleave many different RNA molecules. This feature allows the Bipartite II DNAzyme to selectively cleave RNA in vitro, and it may allow the Bipartite II DNAzyme to selectively cleave RNA in cells, such as bacteria or virus infected cells, or cancer cells, where the RNA, or protein products encoded by the RNA, are implicated in various diseases. Methods of making and using the disclosed enzymes are also disclosed.

### REFERENCE TO RELATED APPLICATIONS

This application claims the benefit of U.S. provisional patent application No. 60/378071, filed May 16, 2002.

### FIELD OF THE INVENTION

This invention relates to RNA-cleaving DNAzymes, methods of making the DNAzymes, and uses of the DNAzymes.

### BACKGROUND OF THE INVENTION

Several classes of catalytic RNA are known to exist in nature. One class has been found as four distinct catalytic motifs, which are named after the ribozymes in which they had originally been found: the hammerhead (Symons 1992), hairpin (Symons 1992), Hepatitis Delta Virus (HDV) (Wu, Lin et al. 1989), and Neurospora VS ribozymes (Saville and Collins 1990). In nature, each of these ribozymes catalyzes a single-turnover, self-cleavage reaction. In the laboratory, however, these motifs have been engineered (via separation of the self-cleaving molecules into distinct “enzyme” and “substrate” entities) to work in trans, i.e. as true enzymatic systems capable of multiple turnovers of substrate (Fauzi, Kawakami et al. 1997), (Guo and Collins 1995), (Hegg and Fedor 1995) and (Stage-Zimmermann and Uhlenbeck 1998). With the exception of the Neurospora VS ribozyme (Rastogi and Collins 1998), the other ribozymes recognize their substrates by Watson-Crick base pairing interactions. The hammerhead and the hairpin ribozymes, in particular, have been modified in the laboratory to be capable of cleaving any substrate RNA sequence site-specifically, and these ribozymes have been successfully used to inactivate a variety of target RNAs in vivo (reviewed (Lustig and Jeang 2001), (Sun, Cairns et al. 2000), (Muotri, Pereira et al. 1999)).

Recently, catalytic DNAs (DNA enzymes or DNAzymes) capable of RNA cleavage have been obtained. Although catalytic DNAs have not been reported in nature, current methods of in vitro selection (“SELEX”) (Tuerk and Gold 1990) from random sequence libraries have permitted the isolation and characterization of a variety of DNAzymes (reviewed in (Breaker 1999)), including those for RNA cleavage. Interestingly, these DNAzymes have been shown to be versatile in terms of cofactor usage for the RNA cleavage reaction (Breaker and Joyce 1994), (Breaker and Joyce 1995), (Faulhammer and Famulok 1996), (Faulhammer and Famulok 1997), (Li, Zheng, et al. 2000), (Roth and Breaker 1998), (Santoro and Joyce 1997), (Geyer and Sen 1997, 1998). Distinct DNAzymes, all capable of cleaving the phosphodiester 3′ to a single ribonucleotide embedded in DNA, have been reported to prefer different divalent cation cofactors, including lead (Breaker and Joyce 1994), magnesium (Breaker and Joyce 1995), calcium (Faulhammer and Famulok 1996), and zinc (Li, Zheng, et al. 2000). One DNAzyme has been reported which requires histidine as cofactor (Roth and Breaker 1998); and, another that works in the absence of any extraneous cofactor (Geyer and Sen 1997), (Faulhammer and Famulok 1997).

The above DNAzymes, all selected specifically to cleave at a phosphodiester immediately 3′ to a single ribonucleotide embedded within a DNA substrate, were either unable to cleave when extended RNA of the same sequence replaced the original DNA substrate (Breaker and Joyce 1994), (Breaker and Joyce 1995), or did cleave, but with significantly reduced rates (Roth and Breaker 1998). Only in a single instance has a DNAzyme (the “8-17” DNAzyme obtained by Santoro and Joyce (Santoro and Joyce 1997)) been reported to cleave comparably well at a single ribonucleotide within a DNA substrate and at an all-RNA substrate (Li, Zheng et al. 2000).

### SUMMARY OF THE INVENTION

This invention relates to RNA cleaving DNAzymes suitable for the sequence-specific cleavage of RNA in trans (the “Bipartite II DNAzyme”). The Bipartite II DNAzyme comprises a conserved catalytic core and substrate binding arms which flank the catalytic core. The substrate binding arms bind to the RNA substrate, and the catalytic core cleaves the substrates.

The catalytic core comprises the sequence 5′-NNNAGGTAGGGGTTCCGCTC-3′ (SEQ. ID NO. 83), wherein N is any nucleotide. The catalytic core preferably comprises the sequence 5′-AGGAGGTAGGGGTTCCGCTC-3′ (SEQ. ID NO. 1). Alternatively, the catalytic core comprises the sequences 5′-TGGAGGTAGGGGTTCCGCTC-3′ (SEQ. ID NO. 2), 5′-TCAAGGTAGGGGTTCCGCTC-3′ (SEQ. ID NO. 3), or 5′-AGAAGGTAGGGGTTCCGCTC-3′ (SEQ. ID NO. 4).

The substrate binding arms preferably comprise 7 nucleotides upstream of the 5′ end of the catalytic core and 7 nucleotides downstream of the 3′ end of the catalytic core. The substrate binding arms can alternatively comprise more than 7 nucleotides downstream of the 3′ end of the catalytic core (for example, 8 nucleotides), or they can comprise less than 7 nucleotides downstream of the 3′ end of the catalytic core (for example, 5 nucleotides).

The target cleavage site in the RNA substrate recognized by the Bipartite II DNAzyme is preferably the sequence 5′-A↓ANNN-3′, where N is any nucleotide. The Bipartite II DNAzyme also recognizes the sequences 5′-A↓UNNN-3′ and 5′-A↓CNNN-3′to a lesser extent. It is also capable of recognizing sequences which contain only 4 nucleotides, such as the sequence 5′-A↓ANN-3′.

The Bipartite II DNAzyme can be altered to bind and cleave different RNA molecules as a substrate by altering the sequence and length of the substrate binding arms so that the substrate binding arms form Watson-Crick base pairs with the substrate. Because the Bipartite II DNAzyme can be modified to bind and cleave a large variety of different RNA molecules, it is potentially useful as a gene therapy tool.

The Bipartite II DNAzyme was selected from a pool of randomized oligonucleotides that were capable of self-cleavage at an internal ribonucleotide (the Bipartite I DNAzymes).

### BRIEF DESCRIPTION OF THE DRAWINGS

In drawings which illustrate embodiments of the invention but which should not be construed to limit the scope of the invention:

FIG. 1A illustrates the design of the starting library structure for the selection of single ribonucleotide phosphodiester-cleaving DNAzymes (SEQ. ID NO. 11).

FIG. 1B illustrates the random-sequence regions of clones selected from cycle 6 (clones numbered 6-61, 6-60, 6-63 and 6-67, Seq. ID Nos. 46 to 49)), and cycle 12 (clones 12-17, 12-29, 12-36, 12-6 and 12-8, Seq. ID Nos. 50-54) of in vitro selection.

FIG. 2 is a graph illustrating the MgCl₂ dependence of clone 12-17.

FIG. 3A shows an electrophoretic gel with a cleavage assay performed with Bipartite I DNAzyme in the presence of different divalent cations as cofactors.

FIG. 3B is a graph illustrating the reaction rates k_{obs} for Bipartite I in the presence of 2 mM Mg²⁺, Mn²⁺, Zn²⁺ and Co²⁺.

FIG. 4 is a graph of the pH versus rate profile for Bipartite I.

FIG. 5A illustrates the design of two randomized libraries for the selection of DNA sequences capable of cleaving extended RNA substrates (SEQ. ID NO. 55).

FIG. 5B illustrates the sequence of clones obtained from re-selection experiments (Seq. ID Nos. 56 to 66).

FIG. 5C illustrates the predicted folded structure for an RNA-cleaving re-selected DNAzyme (SEQ. ID NO. 67).

FIG. 6A is a sequencing gel showing the cleaved product of Bipartite II DNAzyme.

FIG. 6B shows the structure of oligonucleotides used in trans for mapping the cleavage site (Seq. ID Nos. 15 and 24).

FIG. 7A lists the substrate mutants used for Bipartite II DNAzyme for cleavage site identification (Seq. ID Nos. 68 to 82).

FIG. 7B shows an electrophoretic gel with a cleavage assay performed with various substrates containing nucleotide substitutions at the cleavage site, in the presence of Bipartite II enzyme (E1, SEQ. ID NO. 23).

FIG. 7C illustrates the general substrate recognized by the Bipartite II DNAzyme.

FIG. 8 is a graph depicting the reaction rate profile for Bipartite II DNAzyme constructs and substrates derived from the HIV genome.

FIG. 9 is a graph illustrating the multiple turnover profile of Bipartite II DNAzyme with HIV-env substrate.

FIG. 10A illustrates the pH dependence of substrate 9 treated with Bipartite II DNAzyme (E1, SEQ. ID NO. 23) up to pH 8.0 under single-turnover conditions, in the presence of 30 mM MgCl₂ and 10 mM MgCl₂.

FIG. 10B shows the entire pH profile of substrate 9 treated with Bipartite II DNAzyme (E1, SEQ. ID NO. 23) (see Table 3), in the presence of 30 mM MgCl₂.

FIG. 10C shows the Bipartite II DNAzyme (E1, SEQ. ID NO. 23) and substrate (S9, SEQ. ID NO. 18) used for pH profile measurements.

FIG. 11 illustrates the pH versus rate profile in the presence of 2.25 μM (diamonds) or 4.5 μM (squares) of Bipartite II DNAzyme (E1, SEQ. ID NO. 23).

FIG. 12 illustrates the pH dependence of substrate S9 (SEQ. ID NO. 18) and Bipartite II DNAzyme (E1, SEQ. ID NO. 23) complex in the presence of 10 mM MnCl₂ (Δ) and 10 mM CaCl₂ (∇).

FIG. 13 is a pL (pH or pD) versus rate profile of Bipartite II DNAzyme (E1, SEQ. ID NO. 23) and substrate S9 (SEQ. ID NO. 18).

FIG. 14 is a graph depicting the proton inventory of Bipartite II DNAzyme (E1, SEQ. ID NO. 23) and substrate S9 (SEQ. ID NO. 18)

FIG. 15 is a denaturing polyacrylamide gel showing the cross-linked Bipartite II DNAzyme (E1, SEQ. ID NO. 23).

FIG. 16 is an electrophoresis gel depicting cleavage of the dpy-5 mRNA by different constructs of Bipartite II DNAzyme.

### DETAILED DESCRIPTION OF THE INVENTION

Throughout the following description specific details are set forth in order to provide a more thorough understanding of the invention. However, the invention may be practiced without these particulars. In other instances, well known elements have not been shown or described in detail to avoid unnecessarily obscuring the present invention. Accordingly, the specification and drawings are to be regarded in an illustrative, rather than a restrictive, sense.

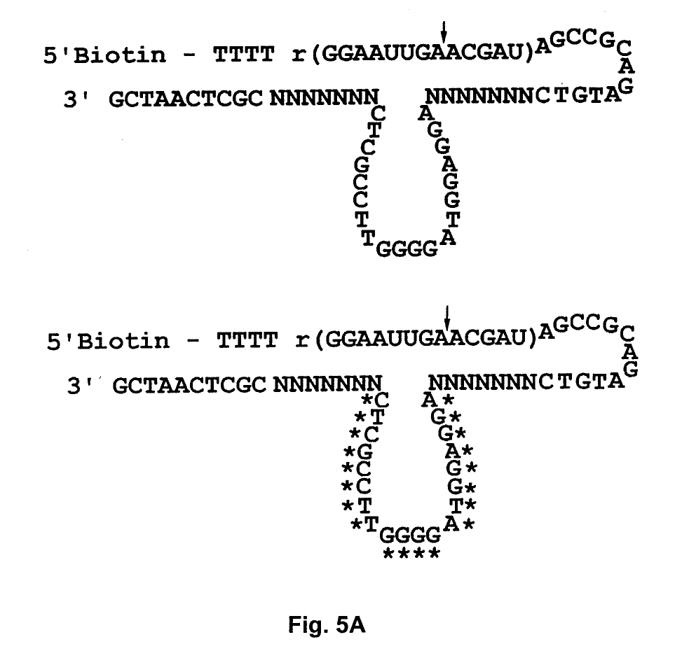

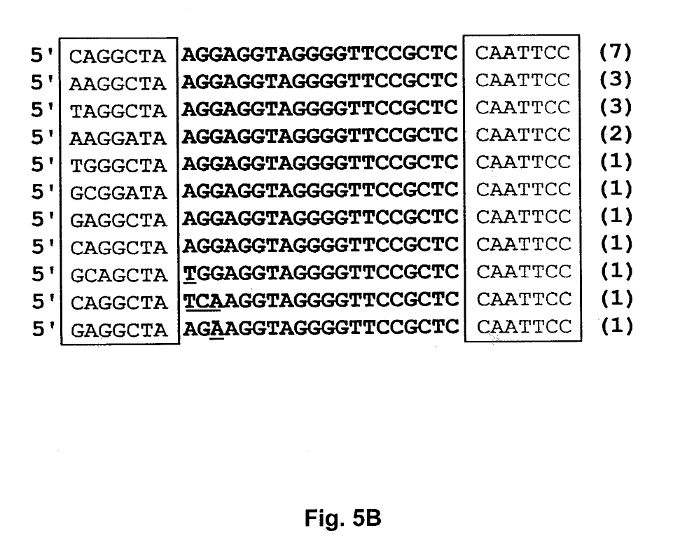

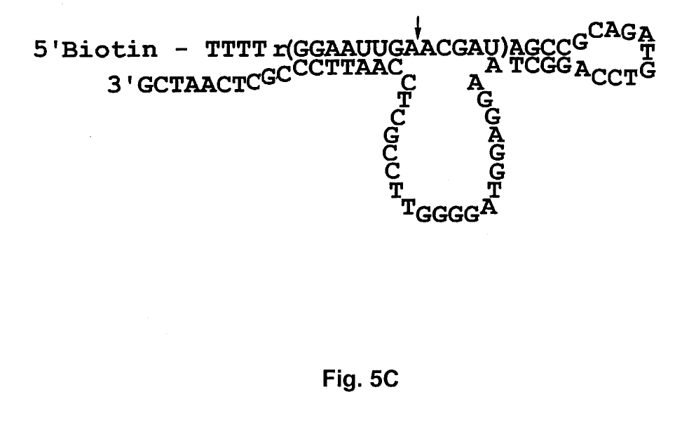

The invention is an RNA-cleaving DNAzyme (the “Bipartite II DNAzyme”), which is capable of cleaving extended RNA substrates in trans. The Bipartite II DNAzyme has a catalytic core that cleaves the target RNA substrates. The catalytic core comprises the general sequence 5′-NNNAGGTAGGGGTTCCGCTC-3′ (SEQ. ID NO. 83), wherein N is any nucleotide. The optimal sequence of the catalytic core is 5′-AGGAGGTAGGGGTTCCGCTC-3′ (SEQ. ID NO. 1). Alternatively, the catalytic core can comprise the sequences:

| | |
|---|---|
| 5′-TGGAGGTAGGGGTTCCGCTC-3′, | (SEQ. ID NO. 2) |
| 5′-TCAAGGTAGGGGTTCCGCTC-3′, | (SEQ. ID NO. 3) |
| or | |
| 5′-AGAAGGTAGGGGTTCCGCTC-3′. | (SEQ. ID NO. 4) |
| (see FIG. 5B) | |

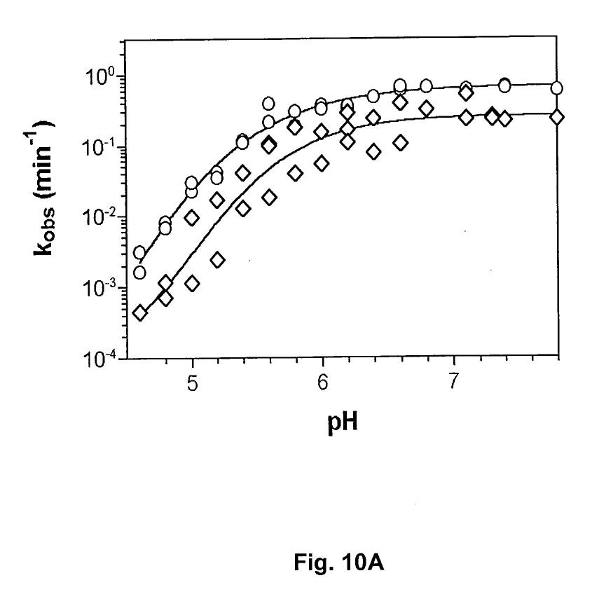

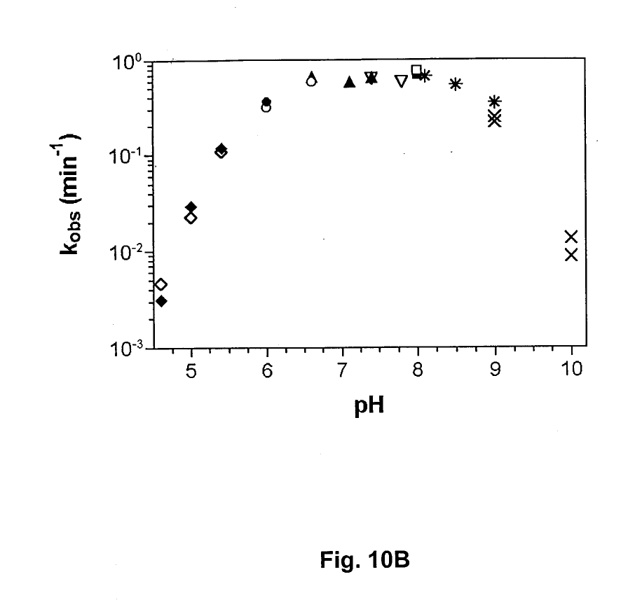

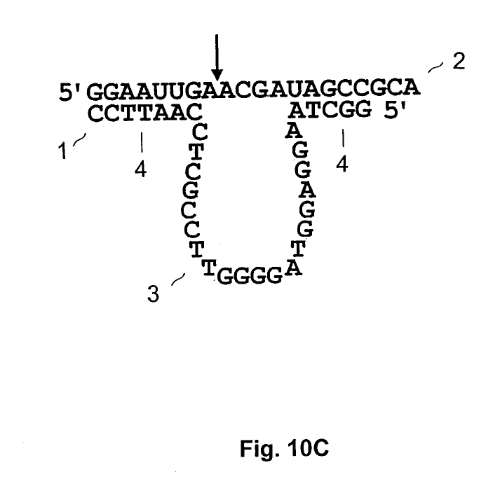

The Bipartite II DNAzyme also comprises substrate binding arms. Referring to FIG. 10C, which illustrates the Bipartite II DNAzyme (1) bound to an RNA substrate (2), the catalytic core (3) is flanked by substrate binding arms (4). The substrate binding arms allow the Bipartite II DNAzyme to bind to different RNA substrates through Watson-Crick base pairing. Once bound to the substrate RNA, the catalytic core of the Bipartite II DNAzyme cleaves the substrate. The sequence and length of the binding arms can be altered to create a Bipartite II DNAzyme that is capable of binding and cleaving any RNA molecule, sequence specifically. The binding arms preferably 7 nucleotides upstream of the 5′ end of the catalytic core and 7 nucleotides downstream of the 3′ end of the catalytic core. The substrate binding arms can alternatively comprise more than 7 nucleotides downstream of the 3′ end of the catalytic core (for example, 8 nucleotides), or they can comprise less than 7 nucleotides downstream of the 3′ end of the catalytic core (for example, 5 nucleotides).

The target cleavage site in the RNA substrate is, preferably, a 5 nucleotide sequence 5′-A↓ANNN-3′ where N can be any nucleotide. The target cleavage site is located between two stems in the substrate, which pair with the substrate binding arms of the Bipartite II DNAzyme through Watson-Crick base pairing. To a lesser extent, the Bipartite II DNAzyme can recognize target cleavage sites with the sequences 5′-A↓CNNN-3′ and 5′-A↓UNNN-3′. Alternatively, the target cleavage site can comprise 4 nucleotides having the sequence 5′-A↓ANN-3′, although shorter cleavage target sites are also recognized to a lesser extent by the Bipartite II DNAzyme.

Because the sequence of the Bipartite II DNAzyme binding arms can be altered to bind to different target RNA substrates through Watson-Crick base pairing, the Bipartite II DNAzyme is capable of recognizing and cleaving a wide variety of different RNA substrates. For example, by altering the sequence of the binding arms flanking the catalytic core, it is possible to synthesize Bipartite II DNAzymes which are capable of cleaving RNA derived from HIV genes tat, rev, nef, and the C. elegans gene dpy-5. The Bipartite II DNAzyme is also capable of cleaving RNA substrates under physiological conditions. Because this DNAzyme is capable of recognizing a variety of different RNA substrates, it may be useful for targeting and inactivating RNA molecules which may be implicated in various diseases, particularly mRNA molecules.

For example, the Bipartite II DNAzyme may be useful in a method for treating cells infected by a virus, as the DNAzyme can be modified to target, bind, and cleave mRNA produced by the virus in the cell. For example, the sequence of the Bipartite II DNAzyme substrate binding arms could be modified to target an mRNA molecule encoded by a viral gene essential for viral replication, cell lysis, production of viral toxins, etc. By cleaving the targeted mRNA, Bipartite II DNAzyme could reduce the viability of the virus and reduce, or even prevent, further infection by the virus. The ability of the Bipartite II DNAzyme to cleave at different ribonucleotide combinations can also be used advantageously to inactivate mRNAs from different stages of the viral life cycle. Moreover, the Bipartite II DNAzyme would be particularly useful against viruses such as HIV or other viruses which undergo mutation at rapid rates.

As another example, the Bipartite II DNAzyme may also be useful in a method for treating cells infected by a bacterium, fungus, protozoan, or other microorganisms. The Bipartite II DNAzyme could be modified to target, bind, and cleave mRNA encoded by genes in the bacterium, fungus, protozoan, or other microorganisms that are necessary for replication, for cell lysis, and for producing toxins or other virulence factors. By targeting the mRNA, Bipartite II DNAzyme could reduce the virulence of the microorganisms or prevent the infection of the microorganisms from spreading. Again, the ability of the Bipartite II DNAzyme to recognize different RNA substrates could be used to target the microorganisms at different stages in the microorganisms' life cycle.

The Bipartite II DNAzyme may also be useful as a gene therapy tool for targeting mRNA in cells produced by the cells themselves, but which are deleterious to the cell. For example, the Bipartite II DNAzyme could be used to target and inactivate niRNA encoded by genes which cause cancer, arthritis, Alzheimer's, or any number of other genes whose gene products cause disease.

The Bipartite II DNAzyme is also useful as a method of cleaving or inactivating RNA substrates in vitro. For example, the Bipartite II DNAzyme may be used for trimming the 3′ ends of in vitro transcribed RNAs into specific sizes.

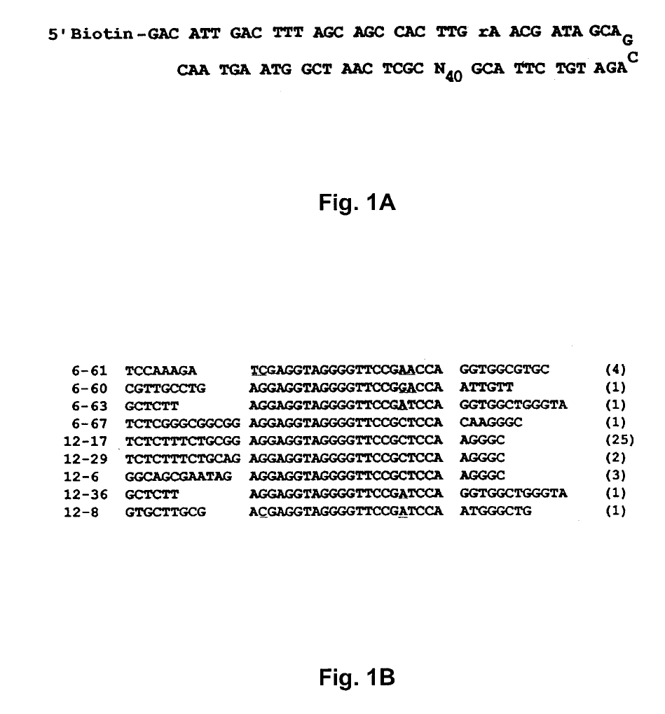

To generate the Bipartite II DNAzyme, the inventors first generated a library of oligonucleotides containing an internal ribonucleotide (see FIG. 1A, SEQ. ID NO. 11). The oligonucleotides were selected for their ability to self-cleave at the internal ribonucleotide. The selected oligonucleotides were amplified by PCR and then re-selected. After twelve rounds of selection, the oligonucleotides were cloned and sequenced. One sequence appeared most abundantly in the clones, which was designated the “Bipartite I DNAzyme” (see FIG. 1B, Seq. ID Nos. 46 to 54).

The Bipartite I DNAzyme has a catalytic core with a general sequence comprising 5′-NNGAGGTAGGGGTTCCGNNCCA-3′ (SEQ. ID NO. 84). Preferably, the sequence of the catalytic core is:

5′-AGGAGGTAGGGGTTCCGCTCCA-3′ (SEQ. ID NO. 5).

Alternatively, other DNAzymes amplified from the pool of self-cleaving oligonucleotides contain a catalytic core with the sequences:

| | |
|---|---|
| 5′-TCGAGGTAGGGGTTCCGAACCA-3′, | (SEQ. ID NO. 6) |
| 5′-AGGAGGTAGGGGTTCCGGACCA-3′, | (SEQ. ID NO. 7) |
| 5′-AGGAGGTAGGGGTTCCGATCCA-3′, | (SEQ. ID NO. 8) |
| 5′-AGGAGGTAGGGGTTCCGATCCA-3′, | (SEQ. ID NO. 9) |
| and | |
| 5′-ACGAGGTAGGGGTTCCGATCCA-3′. | (SEQ. ID NO. 10) |

The Bipartite II DNAzyme was then selected from a randomized pool of Bipartite I DNAzymes having mutations either within the catalytic core or mutations flanking the catalytic core (see FIG. 5A). The DNAzymes in the randomized pool were selected for their ability to cleave extended RNA substrates.

The inventors have also conducted experiments to characterize the Bipartite I and Bipartite II DNAzymes, to characterize the optimal sequences of the target cleavage site of the Bipartite II DNAzyme, and to construct a Bipartite II DNAzyme capable of targeting and cleaving a pre-selected RNA target under physiological conditions.

### EXAMPLES

Without limiting the scope of the invention, the following examples illustrate embodiments of the invention.

### Example 1

In vitro Selection of DNA Sequences Capable of Cleaving at a Single Internal Ribonucleotide (“Bipartite I”)

1×10¹⁴ different oligonucleotides sequences, with ^{˜}5 copies of each sequence, are used to initiate the in vitro selection (Breaker and Joyce 1994). All oligonucleotides were synthesized on a 0.2 μmole scale by the UCDNA, University of Calgary. 30 OD units were purified by preparative polyacrylamide gel electrophoresis (PAGE), the DNA identified by UV shadowing, and eluted into a buffer containing 0.5 M NH₄OAc, 10 mM Mg(OAc)₂, 1 mM Na.EDTA, and 0.1% SDS. Eluted DNA was concentrated on Spice C-18 columns (Analtech), recovered with 30% acetonitrile, and lyophilized. Oligonucleotide concentrations were calculated from measuring absorbances at 260 nm, assuming 1 OD unit equaled 40 μg/ml of single-stranded nucleic acid.

The library of oligonucleotides (see FIG. 1A, SEQ. ID NO. 11, where N₄₀ represents a 40-nucleotide random sequence region), immobilized via a 5′-biotin moiety to a streptavidin column, and after being made single-stranded (each containing a single ribonucleotide within its 5′ fixed sequence) was allowed to fold in 10 mM MgCl₂. Following incubation at room temperature for 1 hour, the column was then flushed with buffer to collect any DNA molecules that may have catalytically cleaved themselves at their internal ribonucleotide's phosphodiester. These molecules were then re-converted to double-stranded DNA and the latter is used to seed a PCR reaction, with a primer incorporating the target ribonucleotide and biotin.

The 10 ml PCR reaction contained 5 nmoles of the primers rWT-bio and WT1, and 2.5 nmoles of primer WT2 with the following sequences:

| | | |
|---|---|---|
| Template library: | 5′-ACGATAGCA GCA GAT GTC TTA CGN₄₀CGC TCA | (SEQ. ID NO. 11) |
| | ATCGGT AAG TAA C | |
| Primer rWT-bio: 5′-biotin-GAC ATT GAC TTT AGC AGC CAC TTG rA | (SEQ. ID NO. 12) | |
| Primer WT2: | 5′-TGA CTT TAG CAG CCA CTT GAA CGA TAG CAG | (SEQ. ID NO. 13) |
| | CAG ATG TCT TAC G | |
| Primer WT1: | 5′-GTT ACT TAC CGA TTG AGC G | (SEQ. ID NO. 14) |

The PCR reactions used were 10 mM Tris.HCl, pH 8.3, 2 mM MgCl₂, 0.2 mM each dNTP, and 0.01 U/μl Taq polymerase. Cycling conditions used were 94° C. for 30 seconds, 52° C. for 30 seconds, and 72° C. for 2 minutes. 15 PCR cycles were used. Unincorporated primers and dNTPs were removed by dialysis in 50 mM Na.HEPES pH 7.4, 0.2 M NaCl. The dialyzed PCR product was loaded in a 5 ml avidin-agarose columns (Sigma), washed with 10 column volumes of 50 mM Na.HEPES, pH 7.4, containing 0.2 M NaCl, followed by 2 column volumes of water. Single-stranded DNA was obtained by washing the column with 2 ml 0.2 N NaOH, followed by 2 ml of water, and 7 column volumes of 50 mM Na.HEPES, pH 7.0. The column was incubated 20 hours at room temperature with 50 mM Na.HEPES pH 7.0, 0.5 M NaCl.

To start the selection, the column was incubated for 1 hour at room temperature with the above buffer plus 10 mM MgCl₂. Eluted DNA was amplified by PCR using primers WT1 (SEQ. ID NO. 14) and WT2 (SEQ. ID NO. 13), and the PCR product gel-purified. Purified DNA was used to seed a new PCR reaction containing primers rWT-bio (SEQ. ID NO. 12) and WT1 (SEQ. ID NO. 14), and following the PCR the amplified DNA was ethanol precipitated and loaded onto a streptavidin column (Genosys™). Subsequent rounds of selection were performed as the first cycle, except that PCR volumes were decreased by 80-fold, and the DNA was precipitated with ethanol following PCR. To increase the stringency of the selection, MgCl₂ concentration was decreased to 1 mM, and selection times were decreased over the cycles (summarized in Table I).

TABLE 1
Selection stringency protocol used for in vitro
selection of self-cleaving DNA molecules.

| Cycles | MgCl₂ (mM) | Time (min) |
|---|---|---|
| 1-5 | 10 | 60 |
| 6 | 1 | 60 |
| 7 | 1 | 15 |
| 8 | 1 | 5 |
| 9-10 | 1 | 1 |
| 11-12 | 10 | 1 |

Twelve cycles of in vitro selection is applied, however, an enrichment of the population of active molecules is observed only after six cycles. Selection stringency is also increased by decreasing the MgCl₂ concentration and the reaction times from 10 mM and 1 hour to 1 mM and 1 minute. By following the twelfth cycle of in vitro selection, the kinetics of self-cleavage of the pool as a whole also fits to a single exponential, suggesting the existence of a single dominant catalytic sequence within the pool, with a k_{obs} rate constant for self-cleavage of ^{˜}0.3 min⁻¹, when incubated in the presence of 2 mM MgCl₂, at 23°.

DNA pools from cycles 6 and 12 were cloned using a TA cloning kit (Invitrogen™), and the resulting recombinant plasmids were purified with a Qiagen™ plasmid purification kit. Plasmid inserts were PCR-amplified with primers WT1 and WT2, purified on 8% native gels, and sequenced with a ThermoSequenase radiolabeled terminator cycle sequencing kit (Amersham™). The results are summarized in FIG. 1B, with all of the sequenced clones containing one copy of a 22-nucleotide conserved sequence element within its N₄₀ region. The number of individual clones obtained for each sequence is indicated on the right. Nucleotide substitutions in the consensus sequence (compared to the sequence of clone 12-17, SEQ. ID NO. 50) are underlined. The overall sequence represented by clone 12-17 (SEQ. ID NO. 50, FIG. 1B) is the most abundant one obtained from the twelfth round of selection (representing 25 out of 32 clones).

### Example 2

Kinetic Assays on Selected DNAzyme Clones

Kinetic assays were carried out on the selected clones. Cloned plasmid inserts were PCR amplified with primers WT1 (SEQ. ID NO. 14) and WT2 (SEQ. ID NO. 13), gel purified, and used to seed new 100 μl PCR reactions containing 100 pmoles of 5′ ³²P-labeled primer rWT (SEQ. ID NO. 12) and 100 pmoles of WT1-bio (SEQ. ID NO. 14). The duplex DNA was loaded onto a streptavidin column (Genosys) and washed with 10 column volumes of 50 mM Na.HEPES, pH 7.4 containing 0.5 M NaCl. The single-stranded DNA (containing a single ribonucleotide) was eluted with 100 μl 0.2 M NaOH, followed by 100 μl H₂O. The eluate was pH neutralized, precipitated with ethanol, gel purified, and stored at −80° C.

A typical kinetic assay was carried out by incubating ^{˜}30 nM DNA in a 20 μl reaction containing 45 mM Na.HEPES, pH 7.4, and 0.15 M NaCl. The DNA was heated to 90° C. for 80 seconds, cooled, and equilibrated at 23° C. for 10 minutes. A “null” aliquot was quenched at time zero with excess EDTA. To start the reaction, MgCl₂ to the appropriate final concentration was added. At each time point the reaction was terminated by dilution in 98% formamide containing 5 mM Tris pH 8.0, 60 mM EDTA, and 0.05% each of xylene cyanol and bromophenol blue. Reaction products were separated in 8% denaturing polyacrylamide gels, and gel bands were quantified with a BioRad Phosphorimager. Observed rate constants (k_{obs}) were obtained from a curve fitted to a plot of fraction cleaved versus time, using a first-order rate formalism, y=x (1−e^{-kt}), where y is the fraction reacted at time t, x is the fraction reacted at the end-point, and k is the observed rate constant. Rate constants were estimated from at least two independent experiments.

The observed rate constants for self-cleavage by the different clone categories obtained from round 12 (all of which contain the consensus sequence, but vary elsewhere within their N₄₀ regions) is summarized in Table 2. Clone 12-17 has a fast (by ribozyme standards) rate constant for self-cleavage, ^{˜}1.7 min⁻¹, in 10 mM MgCl₂ at 23° C. and those whose sequences diverge from that of clone 12-17 display somewhat lower self-cleavage rates. Clone 12-17 is designated the “Bipartite I DNAzyme.”

TABLE 2
Rate constants for intramolecular cleavage of internal ribonucleotide
phosphodiesters by clones selected from the twelfth round of in vitro
selection. Self cleavage assays were performed in 45 mM Na.Hepes
(pH 7.4), 150 mM NaCl, 10 mM MgCl₂, 23° C.

| Clone | k_{obs} (min⁻¹) | Reaction Endpoint |
|---|---|---|
| 12-17 | 1.72 ± 0.08 | 72.82% |
| 12-29 | 1.37 ± 0.08 | 62.31% |
| 12-6 | 0.90 ± 0.05 | 55.65% |
| 12-36 | 0.44 ± 0.03 | 75.18% |
| 12-8 | 0.56 ± 0.03 | 69.52% |

### Example 3

Dependency of Bipartite I DNAzyme on Divalent Metal Salts

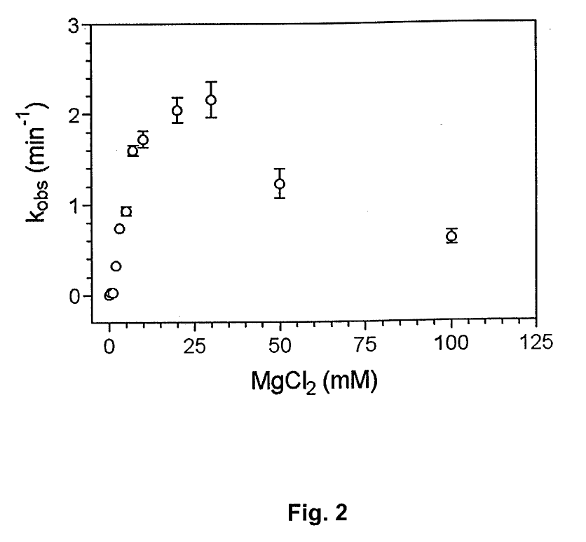

Bipartite I DNAzyme (Clone 12-17) has an absolute requirement for magnesium or certain other divalent metal salts for its self-cleavage. FIG. 2 shows a plot of the magnesium dependence of the observed cleavage rate constant, k_{obs}, of Bipartite I DNAzyme with clear rate increases up to 20 mM Mg²⁺, above which there is a rate decrease, possibly connected with a change in the reaction equilibrium, into a ligation reaction. Self-cleavage reactions were performed in 45 mM Na.Hepes pH 7.4, 150 mM NaCl, 23° C. The Hill coefficient is 2.01±0.39, and K_{D} is 4.5±1.1.

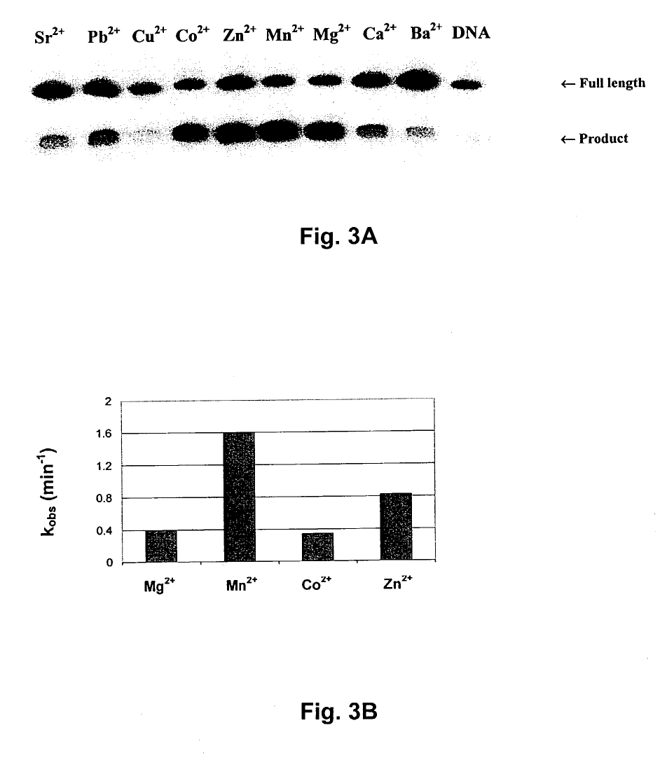

This versatility of divalent metal usage is further explored by incubations in buffer at pH 7.4 in the presence of 1 mM of the chloride salts of, respectively, Mg²⁺, Ca²⁺, Sr²⁺, Ba²⁺, Mn²⁺, Zn²⁺, Cu²⁺, and Co²⁺, and the acetate of Pb²⁺ (FIG. 3A),. This study indicates that only Mg²⁺, Mn²⁺, Zn²⁺, and Co²⁺ support efficient self-cleavage with Mn²⁺ being the best cofactor, with a k_{obs} for self-cleavage measured at 1.62±0.04 min⁻¹ in 2 mM Mn²⁺ (FIG. 3B).

### Example 4

pH-Dependency of the Bipartite I DNAzyme

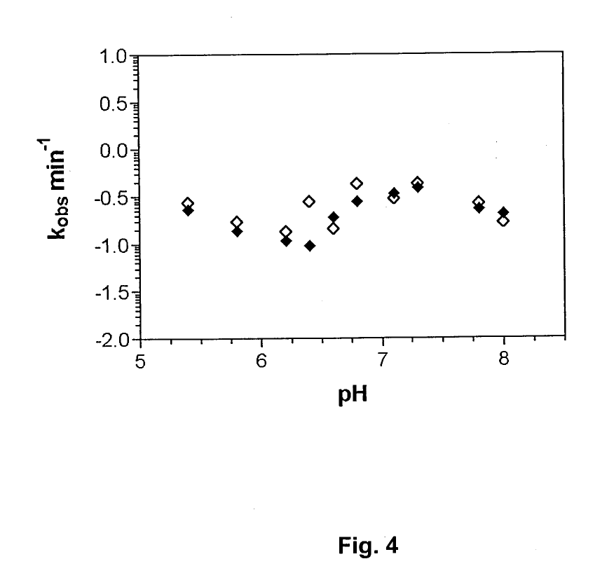

The dependence of k_{obs} on pH was analyzed under single-turnover conditions in the presence of 2 mM MgCl₂. Buffers used were: 45 mM NaOAc, for pH 5.4; 45 mM Na.MES, for pH 5.8-6.6; 45 mM Na.MOPS, for pH 6.8-7.3; 45 mM Na.HEPES for pH 7.4-7.8; 45 mM Tris.HCl for pH 8.0. The experiments indicate that k_{obs} is essentially independent of pH in the range of 5.4-8.0 at 23° C., in the presence of 2 mM MgCl₂, 150 mM NaCl, and 45 mM buffer at the appropriate pH. (See FIG. 4; empty and filled diamonds represent different replicates.)

### Example 5

Randomization and Re-Selection of DNA Sequences for Cleavage of Extended RNA Substrates

It appears that the highly conserved 20-nucleotide motif (with the remaining 2 of the 22 nucleotides of the conserved core implicated in base pairing close to the cleavage site) obtained from the earlier selection of Bipartite I DNAzyme has incorporated a catalytic entity, capable of cleaving RNA phosphodiesters. Hence, two libraries were used to select DNA sequences capable of cleaving extended RNA substrates.

Library A (SEQ. ID NO. 55) contains an intact conserved 20-nucleotide motif while introducing seven fully randomized DNA nucleotides on either side of this conserved core. In addition, a 13-ribonucleotide RNA of the same sequence as the DNA surrounding the single ribonucleotide of the original library, replaces the “substrate” DNA. If the 20-nucleotide core suitable for single ribonucleotide cleavage was not optimal for cleavage at an extended RNA stretch, Library B incorporated, in addition to the basic features of Library A, a partial randomization of the 20-nucleotide catalytic core, to a degeneracy (Breaker and Joyce 1994b) of 12%, such that each individual sequence in this library has 1-3 mutations. (See FIG. 5A: Ribonucleotides are shown in parentheses. N represents the random regions, with equal probabilities of A, T, C or G incorporation. Bases with asterisks were mutagenized to the other 3 nucleotides to the extent of 12%.)

In the reactions, 153 pmoles of template from each library were used to seed a 500 μl primer extension reaction containing 185 pmoles of primer 13RNA-bio [5′biotin-d(TTT T)-r(GGA AUU GAA CGA U)-d(AGC CGC AGA TGT C) (SEQ. ID NO. 15)], 0.08 mM of each dNTP, 10 units of Taq polymerase, and a trace amount of α-P³²-dATP. Cycling conditions were 94° C. for 1 minute, followed by 52° C. for 1 minute, followed by 72° C. for 2 min. The completed reaction was precipitated with ethanol and the RNA-DNA chimera library gel-purified by 8% denaturing PAGE. The DNA was loaded onto 4 streptavidin columns (Genosys), pre-equilibrated with 50 mM Na.HEPES, pH 7.4, containing 0.5 M NaCl. Unbound DNA was eluted with 30 column volumes of binding buffer. Columns were treated, successively, with 100 μl of 0.1 N NaOH , 100 μl H₂O, and 40 column volumes of 50 mM Na.HEPES pH 7.4, containing 0.15 M NaCl. To start the selection, DNA was incubated for 1 hour at 23° C. in the above HEPES buffer supplemented by 10 mM MgCl₂. Eluted DNA was PCR amplified (in 100 μl reaction) with primers WT1 and WT2-short (5′ GCC GCA GAT GTC) (SEQ. ID NO. 16) and gel purified. 10 μl were used seed a new PCR reaction with primers 13RNA-Bio (SEQ. ID NO. 15) and WT1 (SEQ. ID NO. 14). After 7 cycles, selected molecules were cloned and sequenced.

FIG. 5B shows, cumulatively, the sequences of 8 clones analyzed from Library A and 14 clones analyzed from Library B (Seq. ID Nos. 56 to 66). (In FIG. 5B, the two 7 nucleotide random regions (in boxes) surround the catalytic core (unboxed)) The number of clones for each sequence is indicated on the right. Mutations in the catalytic core are underlined.) The selected flanking N₇ elements on either side of the 20-nucleotide core gave consistently conserved sequences, particularly the N₇ stretch 3′ to the conserved core. From library B, only three clones carry mutations within the conserved core (one mutation, three mutations, and one mutation respectively—the last three sequences listed in FIG. 5B). Significantly, all of these mutations are present at the 3′ and 5′ edges of the 20-nucleotide stretch. These data suggest that the 20-nucleotide conserved sequence, as seen in the original Bipartite I DNAzyme (clone 12-17, SEQ. ID NO. 50), is indeed optimal for catalyzing phosphodiester cleavage, whether at a single ribonucleotide or at an extended stretch of RNA. Upon further inspection, it appears that on folding, the NNGGCTA and CAATTCC sequences flanking the conserved 20-mer could base pair to RNA and DNA sequences 5′ and 3′ to the putative cleavage site within the RNA, forming five and seven base-pairs respectively (FIG. 5C, SEQ. ID NO. 67)).

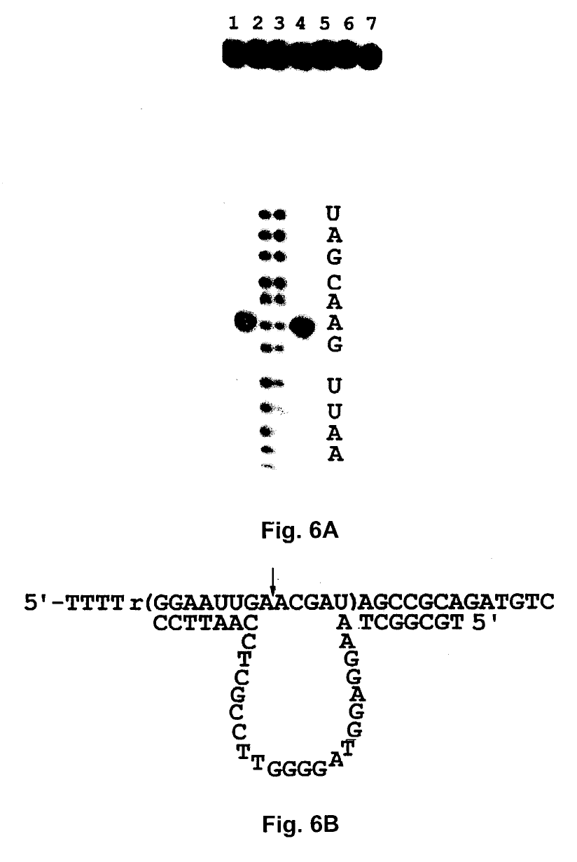

FIGS. 6A and 6B show that the cleavage site on the RNA substrate for the re-selected Bipartite DNAzymes (the “Bipartite II DNAzymes”) remains the same as in the initial selection with a single ribonucleotide. FIG. 6A is a sequencing gel showing the cleaved product of Bipartite II DNAzyme E2 (a version of Bipartite II DNAzyme, see Table 3, SEQ. ID NO. 24). (Lanes (1) and (4)13-RNA (SEQ. ID NO. 15), Bipartite II DNAzyme (E2, SEQ. ID NO. 24), and 30 mM MgCl₂; Lanes (2) and (3) alkaline hydrolysis ladder, from 30 minutes (lane 2) and 25 minutes (lane 3) of incubation; Lane (5) 13-RNA (SEQ. ID NO. 15), Bipartite II DNAzyme (E2, SEQ ID NO. 24), 25 mM EDTA; Lane (6) 13-RNA (SEQ. ID NO. 15), MgCl₂; Lane (7) 13-RNA (SEQ. ID NO. 15). FIG. 6B shows the structure of the oligonucleotides used in trans for mapping the cleavage site. The DNA/RNA chimera substrate 13-RNA (SEQ. ID NO. 15) has 13 ribonucleotides (shown in parentheses). Arrow indicates cleavage site. Bipartite II DNAzyme (E2, SEQ. ID NO. 24) was used in the trans assay. For the alkaline hydrolysis ladder, 1 pmole of 5′ ³²P-labeled 13-RNA was heated at 80° C. in a 20 μl reaction containing 50 mM Na₂CO₃/NaHCO₃ (titrated to pH 9.0) for 25 or 30 minutes. Reactions were neutralized with 40 μl of 0.1 M Na.HEPES, pH 7.4, ethanol precipitated, and analyzed on a 20% PAGE. A trans reaction of the Bipartite II DNAzyme, containing trace amounts of 5′ ³²P-labeled substrate 13-RNA, 2.5 μM DNAzyme E2, and either 30 mM MgCl₂ or 25 mM EDTA, were reacted for 40 minutes, and loaded together on the same gel.

FIG. 6A shows that the cleavage product band runs flush with the alkali-hydrolyzed band of the “cleavage-site” adenosine, suggesting (as in the product of the alkaline hydrolysis) the presence of a 2′, 3′ cyclic phosphate in the DNAzyme-cleaved product. Moreover, the 3′-cleavage fragment of 13-RNA (SEQ. ID NO. 15) is easily labeled with [γ-³²P]-ATP and T4 polynucleotide kinase under conditions where only a free hydroxyl is kinased, consistent with the presence of a free 5′ hydroxyl group on this cleavage fragment.

### Example 6

Substrate Specificity of Bipartite II DNAzyme

As described above, in order to map the cleavage site of the reselected, extended RNA-cleaving DNAzymes, the enzyme and its RNA-DNA chimera substrate were used in trans. The success of that experiment suggested that the basis for substrate recognition by the extended RNA-cleaving Bipartite II DNAzyme is likely as shown in FIG. 6B, with the 7- and 8-nucleotide substrate-binding arms of the enzyme separated by five nucleotides of substrate (5′ A↓ACGA). The observed reaction rate, k_{obs}, under single-turnover conditions, for substrate 7 (SEQ. ID NO. 17) and Bipartite II DNAzyme (E1, SEQ. ID NO. 23) (see Table 3), with enzyme-substrate recognition stems of 7 and 5 base pairs, respectively, is 0.27±0.04 min⁻¹ (in 30 mM MgCl₂, 23° C.). Substrate 7 has the sequence of the chimera RNA-DNA oligonucleotide target for cleavage, 13-RNA used for re-selection, except that it is entirely RNA. Bipartite II DNAzyme (E1, SEQ. ID NO. 23) is the enzyme sequence obtained after re-selection, and it has the consensus sequence in the two binding arms, (it represents, therefore, the first Bipartite II enzyme obtained). A shorter substrate RNA, such as substrate 9 (SEQ. ID NO. 18) (which is 6 ribonucleotides shorter at the 3′ end than substrate 7, but maintains 7+5 base pairs of enzyme-substrate interaction—see Table 3), has a k_{obs} value of 0.66±0.036 min⁻¹ in 30 mM MgCl₂, 23° C. The reaction end point for substrate 7 and 9 were ^{˜}80% cleavage.

TABLE 3

Oligonucleotides used for investigation of the Bi-
partite II DNAzyme (Seq. ID No.s 17 to 31).

| Oligonu- | |
|---|---|
| cleotide | Sequence (5′→3′) |
| Substrate | GGAAUUG A↓ ACGA UAGCC GCA GAU GUC |
| 7 | |
| Substrate | GGAA UUGA ↓ ACGA UAGCC GCA |
| 9 | |
| Substrate | GGAA UUGA ↓ ACGA UAGCC UU |
| 31 | |
| HIV-nef | UUU GCU AUA ↓ AGA UGG GUG GCA |
| substrate | |
| HIV-tat | AGA GCA AGA ↓ AAU GGA GCC AGU |
| substrate | |
| HIV-env | AUG AGA GUG ↓ AG GAG AAA UAU C |
| substrate | |
| E1 | GGCTA AGGAGGTAGGGGTTCCGCTC CAATTCC |
| E2 | TGCGGCTA AGGAGGTAGGGGTTCCGCTC CAATTCC |
| E5 | GGCTA AGGAGGTAGGGGTTCCGCTC TAATTCC |
| E6 | GGCTA AGGAGGTAGGGGTTCCGCTC AAATTCC |
| E7 | GGCTA AGGAGGTAGGGGTTCCGCTC GAATTCC |
| E8 | AAGGCTA AGGAGGTAGGGGTTCCGCTC CAATTCC |
| HIV-nef | TGCCACCC AGGAGGTAGGGGTTCCGCTC ATA GCA AA |
| enzyme | |
| HIV-tat | ACTGGCTC AGGAGGTAGGGGTTCCGCTC CTTGCTCT |
| enzyme | |
| HIV-env | GATATTTC AGGAGGTAGGGGTTCCGCTC CACTCTCA |
| enzyme | |

The DNA template for in vitro transcription of substrate 7 is 5′ GACA TCTG CGGC TA TC GTTC AATT CCCT ATAG TGAG TCGT ATTA (SEQ. ID NO. 85).
Variant mutants of substrate 7 had single nucleotide substitutions in the bold positions.
All versions of Bipartite II DNAzymes utilized have their substrate binding arms underlined.

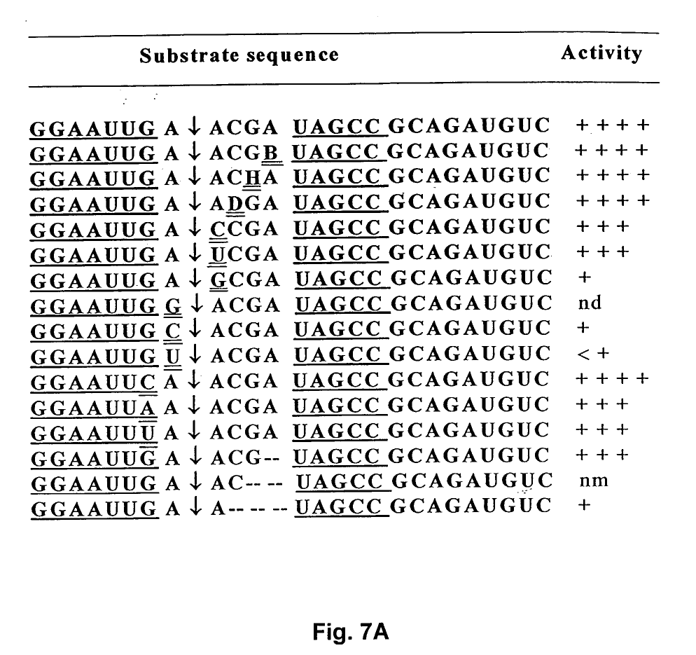

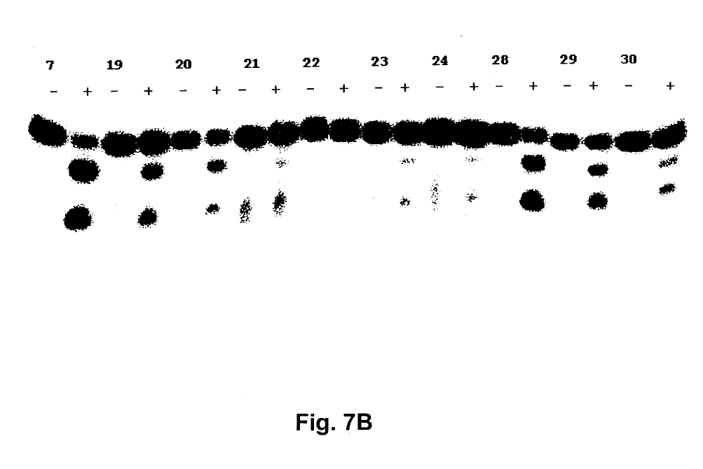

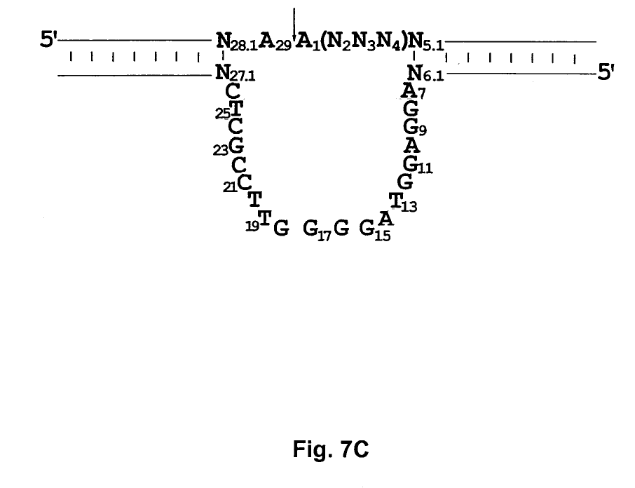

To analyze the nucleotide- and sequence-requirements at the substrate cleavage site and in its immediate environment, a variety of mutants of substrate 7 were designed that deviated systematically from the substrate sequence used for the selection. FIG. 7A lists the substrate mutants used (Seq. ID Nos. 68 to 82). Single underlined ribonucleotides correspond to the base-paired regions. Double underlined ribonucleotides correspond to the mutated position. Arrow indicates the cleavage site. Ribonucleotide B can be C, U or G; H can be A, C, U; D can be A, U, or G. Dashes correspond to deleted positions. Activity measurement were performed in single turnover conditions as described in methods, in the presence of 30 mM MgCl₂. Activity measurements: ++++, k_{obs} in the 10⁻¹ order of magnitude per minute (WT levels); +++, k_{obs} in the 10⁻² order of magnitude per minute; ++, k_{obs} in the 10⁻³ order of magnitude per minute; +, k_{obs} in the 10⁻⁴ order of magnitude per minute; <+, k_{obs} lower than 10⁻⁴ min⁻¹. nd: no detectable cleavage. Bipartite II DNAzyme (E1, SEQ. ID NO. 23) was used. FIG. 7B shows an electrophoretic gel with a cleavage assay performed with various substrates containing nucleotide substitutions at the cleavage site, in the presence of Bipartite II enzyme (E1, SEQ. ID NO. 23) to illustrate the effectiveness of the substrates for the Bipartite II DNAzyme under single turnover conditions. Assay conditions were: 50 mM MgCl₂, during 5 hours of incubation. Substrates used were 7, 19, 20, 21, 22, 23, 24, 28, 29, 30. For each substrate reaction (+), a control without MgCl₂ (−) was performed. The sequences of the five unpaired ribonucleotides for each substrate were: S7 (A↓ACGA), S19 (A↓CCGA), S20 (A↓UCGA), S21 (A↓GCGA), S22 (G↓ACGA), S23 (C↓ACGA), S24 (U↓ACGA), S28 (A↓ACG--), S29 (A↓AC-- --), S30 (A↓A-- -- --).

FIG. 7C illustrates the general substrate recognized by the Bipartite II DNAzyme. The numbering system adopted herein for the core nucleotides of the Bipartite II DNAzyme is analogous to the one proposed for the hammerhead ribozyme (Hertel, Pardi et al. 1992). Single ribonucleotide substitutions at each of the 5 unpaired substrate nucleotides between the two enzyme-substrate stems were analyzed (a total of 15 mutants). In addition, the putatively base-paired position (position 28) immediately 5′ to the A↓A cleavage site was examined. Finally, the question was asked: Is there a requirement for five unpaired nucleotides between the two enzyme substrate stems? Mutant substrates were in vitro transcribed, with 2, 3, and 4 ribonucleotides, respectively, separating the two stems. All of the single turnover assays for substrate mutants were performed with a (7+5) binding arm enzyme (Bipartite II DNAzyme (E1, SEQ. ID NO. 23)—see Table 3). When substrate position 28.1 (FIG. 7B) was mutated, Watson-Crick base-pairing partners were used at positions 27 for the enzyme constructs (E5, E6, and E7—Table 3).

It was found that positions 2, 3 and 4 could be replaced by any ribonucleotide or, indeed, deleted. The presence of five unpaired ribonucleotides (positions 29, 1-4) resulted in the highest k_{obs} values. The loss of one of these unpaired ribonucleotides resulted in a ^{˜}25-fold decrease in k_{obs}, and the loss of three (i.e. two left) resulted in a ^{˜}430-fold decrease. Position 1 could be A, U, C, or G, although the most optimal is A. Positions 28.1 and 27.1 could be replaced by any other base-pair. Position 29 appeared the most critical for recognition by the enzyme. The best cleavage site, in fact, is the one selected (5′A↓A). An A→U and A→C substitutions are tolerated at position 29. No cleavage is observed with a 29A→G mutation. The hybridizing arms could be replaced by any desired sequence, as long as the enzyme recognized substrate through Watson-Crick base pairing.

### Example 7

Kinetic Analyses of Bipartite II DNAzyme

For single-turnover experiments, a large excess of DNAzyme (2.5 μM) was used with trace amounts of 5′ ³²P-labeled substrate. Substrate and DNAzyme were heated together in 45 mM Na.HEPES, pH 7.4, at 90° C. for 1 minute, spun briefly in a microcentrifuge, and equilibrated at 23° C. for 10 minutes. An aliquot was quenched at time zero with excess EDTA. To start the reaction, MgCl₂ of the appropriate final concentration was added. Aliquots were collected at different time intervals and quenched with excess EDTA. Rate constants did not change when the DNAzyme concentration was raised from 2.5 μM to 11.5 μM, suggesting that the substrate present was indeed saturated by the DNAzyme. A control, in which substrate and DNAzyme were heated separately, equilibrated at 23° C. with MgCl₂, and mixed together to initiate the reaction, gave indistinguishable rate constants from those observed using the standard protocol. Cleavage was followed to completion, and the resulting time points fit to the same equation as used for the self-cleavage experiments (see above). Rate constants were determined from at least 2 independent experiments. Substrate mutants were transcribed from DNA templates using T7 RNA polymerase. k_{obs} values obtained with chemically synthesized substrate 9 RNA (SEQ. ID NO. 18) (Dharmacon Research) gave results that were indistinguishable from those obtained using in vitro transcribed substrate 9 RNA (SEQ. ID NO. 18).

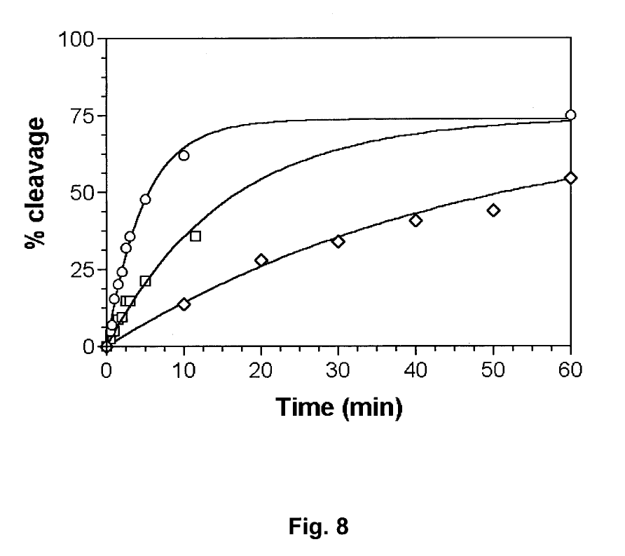

For multiple turnover experiments, HIV-env substrate (see Table 3) RNA (Dharmacon Research) was 5′ end-labelled with T4 kinase and gamma-³²P-ATP, and the labelled RNA size-purified by gel electrophoresis. Excess unlabelled RNA was combined with the labelled RNA to a final volume of 40 μl, and its concentration was estimated by OD measurements. Different volumes of substrate from the above stock were then delivered into each reaction solution. Substrate concentrations tested were 90-6318 nM, and the enzyme concentration was fixed at 22.5 nM. Reaction conditions were: 45 mM Na.HEPES, pH 7.4, 30 mM MgCl₂, 37° C., 0.01% SDS, 20 μl. The data were fit to the Michaelis-Menten formalism using the GraphPad (Prism) software. FIG. 8 is a graph depicting the reaction rate profile for Bipartite II DNAzyme constructs and substrates derived from the HIV genome. Circles represent the design of HIV-env; squares, HIV-tat and diamonds on HIV-nef. k_{obs} was faster in the order: HIV-env>HIV-tat>HIV-nef, , at 23° C. and 30 mM MgCl₂. FIG. 8 demonstrates that Bipartite II DNAzyme is indeed a general DNAzyme, able to accept different RNA substrates, as the active substrate/enzyme constructs had k_{obs} in the order HIV-env>HIV-tat>HIV-nef.

Table 4 summarizes that under single turnover conditions for an in trans cleavage reaction. Bipartite II DNAzyme (E1, SEQ. ID NO. 23) with 7+5 binding arms had a better k_{obs} value than 7+8 arms (enzyme version E2 (SEQ. ID NO. 24)—see Table 3) for substrate 9 (SEQ. ID NO. 18) (0.66 min⁻¹ vs 0.01 min⁻¹, respectively, at 30 mM MgCl₂, 23° C.), although reaction rates were comparable at 37° C. This appears counter-intuitive. However, an analysis of the probable folding of the enzyme E2 containing these particular sequences in its 7+8 arms shows that it likely forms an alternatively folded structure (data not shown). In confirmation of this hypothesis, substrate S31 (SEQ. ID NO. 19) with a similar sequence to S9 (SEQ. ID NO. 18) but having a different 3′ end was tested with Bipartite II DNAzyme E8 (SEQ. ID NO. 28) which has 7+7 binding arms. The 7+7 binding arms in E8 restored the catalytic rate of the enzyme (a k_{obs} value of 0.6 min⁻¹, in 30 mM MgCl₂, 23° C.—see Table 4). Therefore, depending on the sequence of the substrate and the sequence of the binding arms, the length of the binding arms could comprise more than 7 nucleotides.

TABLE 4

Effect of the length of substrate recognition helices on substrate 9.

| | | | | |
|---|---|---|---|---|
| | | Arm | | |
| Substrate | Substrate Sequence | Length | k_{ob} (min⁻¹) 23° C. | k_{ob} (min⁻¹) 37° C. |

| | | | | | |
|---|---|---|---|---|---|
| S9 | GGAAUUGA↓ACGAUAGCCGCA | 7 + 5 | 0.68 ± | 0.04 | 0.19 ± 0.016 |
| S9 | GGAAUUGA↓ACGAUAGCCGCA | 7 + 8 | 0.01 ± | 0.0009 | 0.26 ± 0.003 |
| S31 | GGAAUUGA↓ACGAUAGCCUU | 7 + 7 | 0.61 ± | 0.04 | Not measured |

Rate constants, k_{obs}, were analyzed under single-turnover conditions.
Underlines regions correspond to base-paired positions.
Arrows indicate the cleavage site. (Seq. ID Nos. 18 and 19).

Under single turnover conditions, it was also found that the reaction rate is not affected by the order of addition of reagents. Results from independent experiments in which the reaction is started by addition of magnesium or by mixing enzyme and substrate together suggests that the association step is not rate-limiting.

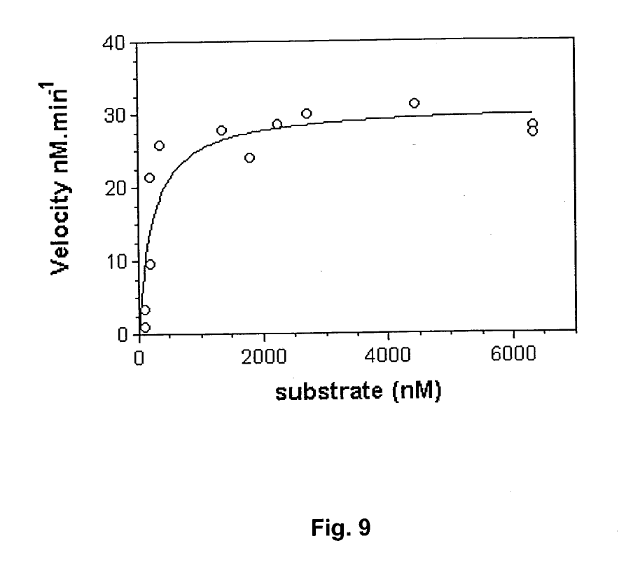

The RNA cleavage reaction of the Bipartite II DNAzyme under multiple substrate turnover conditions with a small RNA substrate derived from the HIV-env gene are presented as the multiple-turnover data in FIG. 9. Substrate concentrations tested were 90-6318 nM, and the enzyme concentration was fixed at 22.5 nM. Reaction conditions were: 45 mM Na.HEPES, pH 7.4, 30 mM MgCl₂, 0.01% SDS, 37° C., 20 μl. In the presence of 30 mM MgCl₂ and at 37° C., k_{cat} is measured as 1.37±0.12 min⁻¹ and K_{M} is 232±93 nM. The reaction rate is therefore in the range of the one obtained with the 10-23 DNAzyme (under similar experimental conditions), and with naturally occurring ribozymes (see Table 5). It therefore represents a valuable and efficient enzyme, with fast reaction rates and with a catalytic efficiency of k_{cat}/K_{M} is 6.0×10⁸ M⁻¹. min⁻¹.

TABLE 5
Comparison of the kinetics of the artificial DNAzymes with the
naturally occurring ribozyines. Sub means substrate, which
could be an extended ribonucleotide (Ext) or a single
ribonucleotide (Sin). T is for temperature. The reaction rate
keicave reported is the rate for cleavage. Assay relates to
multiple-turnover (excess substrate) measurements (MT) or
single-turnover (excess enzyme) measurements (ST). nm = not
mentioned in the referred publication.

| | | | T | | K_{cleave} | | | |
|---|---|---|---|---|---|---|---|---|
| Enzyme | Sub | Metal Ion | (° C.) | pH | (Min⁻¹) | Assay | K_{M} | Reference |
| Bipartite II | Ext | 30 mM Mg²⁺ | 37 | 7.4 | 1.4 | MT | 230 nM | Feldman & Sen |
| | | | | | | | | 2001 |
| 10-23 | Ext | 25 mM Mg²⁺ | 37 | 7.5 | 0.48 | MT | 1.35 μM | He et al., 2002 |
| 10-23 | Ext | 50 mM Mg²⁺ | 37 | 8.0 | 3.4 | MT | 0.76 nM | Santoro & Joyce |
| | | | | | | | | 1997 |
| 8-17 | Ext | 100 μM Zn²⁺ | 23 | 6.0 | 0.064 | ST | 1.1 μM | Li et al., 2000 |
| 8-17 | Ext | 3 mM Ca²⁺ | 37 | 7.4 | 0.04 | ST | N/M | Peracchi, 2000 |
| Hammerhead | Ext | 10 mM Mg²⁺ | 25 | 7.5 | 1.4 | MT | 49 nM | Fedor et al., 1992 |
| Hairpin | Ext | 10 mM Mg²⁺ | 37 | 7.5 | 2.1 | MT | 30 nM | Hampel and Tritz, |
| | | | | | | | | 1989 |
| Neurospora | Ext | 5 mM Mg²⁺ | 30 | 8.0 | 0.7 | MT | 130 nM | Guo & Collins, |
| | | | | | | | | 1995 |
| HDV | Ext | 10 mM Mg²⁺ | 37 | 8.0 | 0.91 | ST | 110 nM | Shih & Been, |
| | | | | | | | | 2000 |
| Pb²⁺ | Sin | 1 Mm Pb²⁺ | 23 | 7.0 | 1 | MT | 2 μM | Breaker & Joyce, |
| | | | | | | | | 1994 |
| Mg²⁺ | Sin | 10 mM Mg²⁺ | 23 | 7.0 | 0.039 | MT | 13 μM | Breaker & Joyce, |
| | | | | | | | | 1995 |
| HD2 | Sin | 100 mM hist | 23 | 7.5 | 0.2 | ST | N/M | Roth & |
| | | | | | | | | Breaker, 1998 |
| Ca²⁺ | Sin | 10 mM Ca²⁺ | 37 | 7.0 | 0.1 | MT | 6.4 μM | Faulhammer et |
| | | | | | | | | al.,1996 |
| Na8 | Sin | 500 mM Na⁺ | 25 | 7.0 | 0.0067 | ST | N/M | Geyer & Sen, |
| | | | | | | | | 1997 |
| 9₂₅-11 | Sin | 200 mM Na⁺ | 37 | 7.4 | 0.044 | ST | N/M | Perrin et al., 2001 |
| 16.2-11 | Ext | 10 μM Zn²⁺ | 37 | 7.5 | 1.4 | MT | 100 nM | Santoro et al., |
| | | | | | | | | 2000 |

### Example 8

Magnesium and Metal Usage by the Bipartite II DNAzyme

The Bipartite II DNAzyme does not have the ability to use zinc and cobalt (tested in different buffers and pH values), but does have the ability to use calcium more efficiently than Bipartite I DNAzyme. Interestingly, the rate constants, k_{obs}, at 10 mM MnCl₂ and at 10 mM MgCl₂ (measured at 23° C., under single-turnover conditions) are 0.14±0.006 and 0.24±0.013, respectively. Whereas Bipartite I has shown a significant preference for manganese (4-10 fold, depending on the buffer, see FIG. 3B) the Bipartite II enzyme, with its RNA substrate, utilizes both manganese and magnesium comparably well (as illustrated in Table 6).

### Example 9

pH Dependence of the Bipartite II DNAzyme

The dependence of k_{obs} on pH was analyzed under single-turnover conditions for a complex of substrate 9 and Bipartite II DNAzyme (E1, SEQ. ID NO. 23) in the presence of 30 mM MgCl₂ (o) and 10 mM MgCl₂ (⋄) at 23° C. (see FIG. 10A, see also FIG. 10C for structure). For FIG. 10B, reactions were carried out in the following conditions: 45 mM NaOAC/Acetic acid for the pH range 4.6-5.8 (,); 45 mM Na.MES, pH 5.8-6.6 (,); 45 mM Na.MOPS, pH 6.6-7.4 ( ); 45 mM Na.HEPES, pH 7.4-7.8 (∇); 45 mM Tris.HCl, pH 8.0 (,); 45 mM Na.EPPS, pH 8.1-9.0 (*); 45 mM Na₂CO₃/NaHCO₃, pH 9.0-10.0 (X). Buffers with overlapping buffering capacity are employed to span the pH range examined. FIGS. 10A and 10B show that the log of k_{obs}, plotted against pH, increased linearly with pH up to pH ˜5.8, at which point it becomes relatively independent of pH, with a k_{obs} maximum of 0.68±0.02 min⁻¹. The data being fit to the equation k_{obs}=kₘₐₓ/[1+10^{(pKa-pH)}], yield a pKa value of 6.0±0.06 (FIG. 10A). The linear portion of the fit has a slope of 1.025±0.27, indicating that a single deprotonation has occurred. At pH 9.0, k_{obs} decreases to 0.237 min⁻¹ (FIG. 10B).

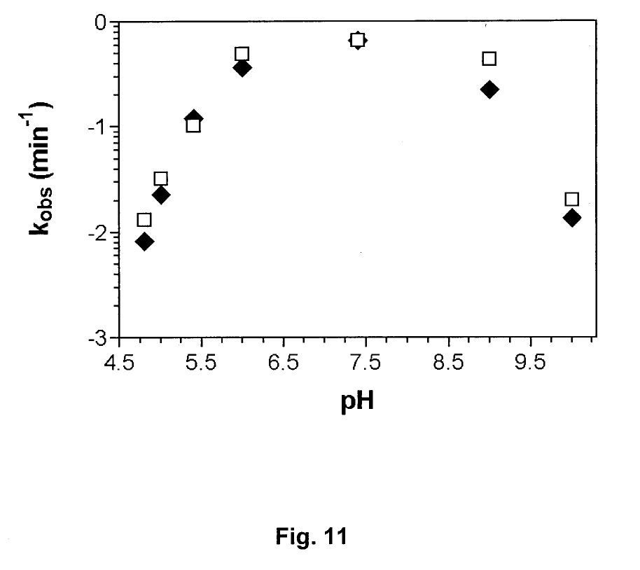

Increasing the concentration of DNAzyme from 2.25 μM to 4.5 μM does not change the pH versus rate profile indicating that the DNAzyme is indeed in large excess (and saturating) with respect to substrate throughout the entire pH range (see FIG. 11, pH versus rate profile in the presence of 2.5 μM (diamonds) or 4.5 μM (squares) of Bipartite II DNAzyme (E1, SEQ. ID NO. 23). Reaction rates were measured in the presence of 30 mM MgCl₂).

### Example 10

Divalent Metal Ion Specificity of the Bipartite II DNAzyme

The divalent metal ion specificity of the Bipartite II DNAzyme (E1, SEQ. ID NO. 23) was analyzed, as described in single turnover assays, in the presence of 45 mM Na.Hepes pH 7.4, and 10 mM metal cofactor (MgCl₂, MnCl₂, and CaCl₂). Table 6 summarizes the results. Reactions were incubated during ˜17 hours, with the metal cofactors. Cleavage reactions using substrate S9 (SEQ. ID NO. 18) were analyzed by PAGE. The cations tested were: Sr²⁺, Ca²⁺, Ba²⁺, Mn²⁺, Co²⁺, Zn²⁺, Cd²⁺, Cu²⁺, Pb²⁺ and Mg²⁺. Because there is an effect of the buffer nature on the metal ion specificity of the 10-23 DNAzyme (Santoro and Joyce 1998), the above reaction were also tested in 50 mM Na.MES pH 6.6, and 50 mM Na.EPPS, pH 8.1.

Different from Bipartite I enzyme, the Bipartite II enzyme was able to use Ca²⁺, in addition to Mg²⁺, Mn²⁺, but does not have the ability to use Zn²⁺ and Co²⁺. The reaction rate constant k_{obs} for cofactors Ca²⁺, Mg²⁺, and Mn²⁺, are shown in Table 6.

TABLE 6
Reaction rates (k_{obs}), min⁻¹, analyzed
under single turnover conditions for Bipartite
II DNAzyme in the presence of 45 mM Na.Hepes
pH 7.4, 10 mM Metal Chloride.

| Metal ion | MgCl₂ | MnCl₂ | CaCl₂ |
|---|---|---|---|
| k_{obs} (min⁻¹) | 0.24 ± 0.013 | 0.14 ± 0.006 | 0.02 ± 0.001 |

### Example 11

Analysis of Bipartite II DNAzyme Cleavage Reaction in the Presence of High Concentrations of Monovalent Cations Alone

The ability of the Bipartite II DNAzyme (E1, SEQ. ID NO. 23) to cleave substrate RNA (S9, SEQ. ID NO. 18) was analyzed in the presence of monovalent cations only. 50 mM HEPES buffer was used for the cleavage assay. Buffers were titrated to appropriate pH with the respective salt hydroxide, after addition of monovalent salts (3M) and EDTA (20 mM). Cleavage assays were performed in a standard single turnover condition, as described. Bipartite II DNAzyme to a concentration of 2.25 μM and ^{˜}30 nM substrate were combined in 10 μl H₂O, heated to 90° C. during 1 minute, and equilibrated at 23° C. Samples were dried under vacuum, and ressuspended in the buffer containing the monovalent salt, during 17 hours.

When Bipartite II DNAzyme and substrate were combined with separated buffer components, in which Na.HEPES was titrated alone to pH 7.4, to result in the final concentration of HEPES, monovalent and EDTA, the percentage of substrate cleavage observed over the same period of time did not change.

For the cleavage rate constant measurement in the presence of 3M LiCl, a similar method was employed, except that aliquots were taken at specific time intervals (over two weeks of incubation). The data was fit to the equation y=x (1−e^{-kt}), as described above.

The reaction catalyzed in the presence of 3M LiCl had a rate constant k_{obs} of ^{˜}1.76×10⁻⁵ min⁻¹, which is ^{˜}40000 fold slower than the optimized reaction in the presence of 30 mM MgCl₂. The end-point of the reaction resulted in cleavage of ^{˜}25% substrate. The reaction contained 20 mM EDTA, which eliminates the possibility that contaminating magnesium could be responsible for stimulating cleavage.

Lower concentrations of LiCl were tested for cleavage by the DNAzyme. About 17 hours incubation in the presence of 2M LiCl (plus 20 mM EDTA), resulted in only ^{˜}3% substrate cleavage. No cleavage was observed in the presence of 0.5 or 1M LiCl.

### Example 12

pH Dependence of the Bipartite II DNAzyme in the Presence of Different Divalent Metal Ion Cofactors

The conditions used for the pH dependence experiments were the same as used in example 9, except that 10 mM CaCl₂ and 10 mM MnCl₂ (final concentrations) were used to initiate the reactions.

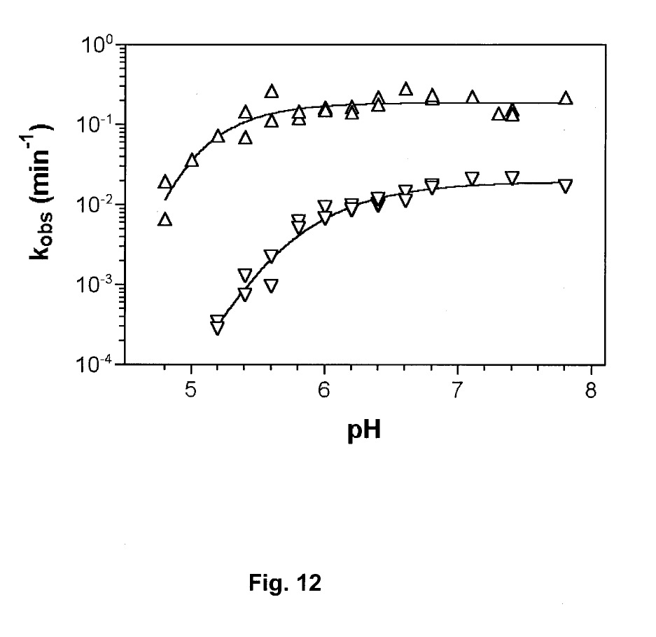

This experiment was performed to investigate whether the pH versus rate profile changes with different cofactors for the reaction. Divalent cations differ in several aspects, including ionic radius, the first pKₐ of a water molecule coordinating to the metal ion, and coordination number. Water molecules bound to a metal ion can be substantially more acidic than free water. Hydroxide ions bound to a metal ion can participate in general acid or base chemistry, and when coordinated to other ligands, the pKₐ of a bound water can be affected (Gesteland, Cech et al. 1998). If the metal ion plays a catalytic role in the reaction, it could be possible that the apparent pKₐ changes from one cofactor to the other, according to their unperturbed pKₐₛ. The unperturbed pKₐ Mg²⁺(OH₂), Mn²⁺(OH₂), and Ca²⁺(OH₂) is 11.4, 10.6 and 12.6, respectively (Table 7). FIG. 12 shows the pH versus rate profile of substrate S9 (SEQ. ID NO. 18) with Bipartite II DNAzyme (E1, SEQ. ID NO. 23) in the presence of 10 mM CaCl₂ and 10 mM MnCl₂.

TABLE 7
First pKa of different Metal(OH₂)complexes.

| Metal ion | First pKa |
|---|---|
| Manganese | 10.6-10.9 |
| Magnesium | 11.4-12.8 |
| calcium | 12.6-13.4 |

The observed pKₐ in the presence of 10 mM MnCl₂ is 5.39±0.17, while in 10 mM CaCl₂ it is 6.24±0.09. The pKₐ measured in the presence of Mg²⁺ is 6.0 (FIG. 12). The Mg²⁺ to Mn²⁺ pKₐ is 0.61 pH units, and the Ca²⁺ to Mg²⁺ pKₐ is 0.23 pH units. A shifted pKₐ of 0.61 pH units would be expected if a manganese-hydroxide is involved in the cleavage chemistry—the pKₐ difference between the unperturbed pKₐ of Mg²⁺(OH₂) (which is 11.4), and Mn²⁺(OH₂), (which is 10.6), is 0.8, (Table 7). Overlapping pHs span the first pKₐs of magnesium and calcium hydroxides (Table 7). That could explain the small pKₐ shift observed in the presence of calcium.

If the pH versus rate profile represents a reaction that is general base catalyzed, and a metal-hydroxide is the general base, then the measured pKₐ should shift to a more acidic or basic pH, according to the choice of divalent cation used. If the rate determining-step of the reaction is indeed chemistry, then one would expect the pKₐ shift observed when the metal ion is manganese (instead of magnesium).

### Example 13

Kinetic Solvent Isotope Effect Experiments on Bipartite II DNAzyme

Information about the extent and nature of the bond making and breaking steps in the transition state may sometimes be obtained by studying the effects of isotopic substitution on the reaction rates. The cleavage of a C—D bond is slower than that of a C—H bond (Fersht 1999). Solvent isotope effects are found from comparing the rates of a reaction in H₂O and D₂O. They are usually the result of proton transfers between electronegative atoms (such as O, N) accompanying the bond making and bond breaking steps in the reaction (Fersht 1999). In order to investigate whether the observed pKₐ (obtained in the pH versus rate profile) is a kinetic pKₐ (a change in the rate determining step of the reaction) (Fersht 1999), or whether it represents the titration of a general base, k_{obs} was measured in the presence of deuterium oxide containing buffer. If the measured pKₐ was a kinetic pKₐ, then the pD profile should be similar to the pH profile. However, if a proton transfer is involved in the reaction mechanism, (as in general base-catalysis), then the reaction rate in D₂O containing buffers should be slower than in aqueous buffers.

For the KSIE experiments, trace amounts of labeled substrate S9 (SEQ. ID NO. 18) was combined with 50 pmoles Bipartite II DNAzyme (E1, SEQ. ID NO. 23), and dried together under vacuum. The pellet was resuspended in 50 mM D₂O containing buffers. A null aliquot was taken for time zero, and the final buffer concentration was 45 mM. Kinetics were performed under single turnover conditions as described earlier, except that the reaction was started by the addition of 30 mM MgCl₂ in 100% D₂O. All solution and buffers were prepared in 99.9% D₂O. Buffers were prepared by directly resuspending the powder in D₂O, and the final pD was determined by adding 0.4 to the pH meter reading (Schowen and Schowen 1982). The titration was performed in less than 5 minutes for each buffer, while stirring, to avoid exchange with atmospheric moisture (and thus loss of deuterium content). Four minute exposures of buffers while being titrated to final pD results in negligible contamination of the D₂O containing solution (Schowen and Schowen 1982). The buffers used to span the pH range were: Na.MES, pH 5.1-6.7; Na.MOPS, pH 6.8-7.2; Na.HEPES, pH 7.4-7.6; Na.EPPS, pH 8.0. Buffers were titrated with sodium deuteroxide, 99 atom % Deuterium atom (Sigma).

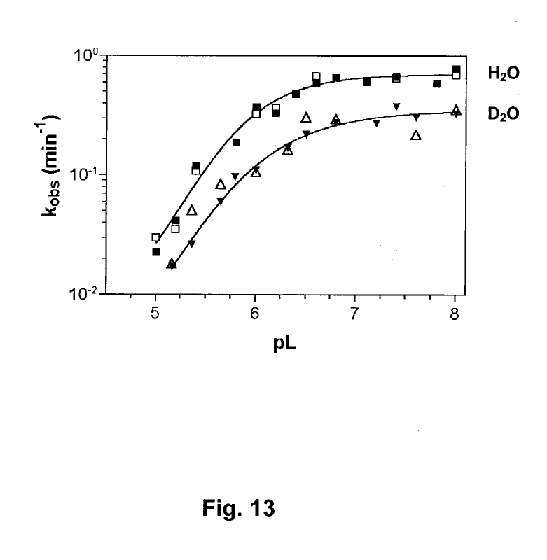

FIG. 13 shows the results of k_{obs} when using deuterium oxide containing buffers throughout the entire pL (pH or pD) range. Squares represent the H₂O containing buffer. Triangles represent the D₂O containing buffer. The pL profile was performed in the presence of 30 mM MgCl₂. The pKₐ measured in H₂O was 5.95±0.06, and in D₂O was, 6.267±0.06. The rate constant in the plateau region of the profile was 0.68±0.02 min⁻¹ and 0.3630±0.014 min⁻¹ in H₂O and D₂O, respectively. The KSIE which is k_{obs}H₂O/k_{obs}D₂O was 1.87, as determined from the plateau region of the profile.

The results from the KSIE experiments show that the reaction rate is lowered throughout the entire pD profile, indicating that the reaction is catalyzed by a mechanism involving general base catalysis. Therefore, the measured pKₐ reflects a real ionization (Nakano, Chadalavada et al. 2000). Also, the inflection point of the curve (the pKₐ) changed to more basic in the pD profile. Failure of the pH-rate profile to behave in this fashion may be an indication that structural changes are being induced by deuteration, or that the inflections result from other mechanistic features than ionization (Cleland, O'Leary et al. 1977).

### Example 14

Proton Inventory

A useful method for determining the number of proton transfers in the transition state is the proton inventory technique. This method relies in measuring the rate constant in H₂O/D₂O mixtures and examining the resulting curves. This can help resolve whether one or two proton transfers occur in the rate-limiting step (Nakano and Bevilacqua 2001).

The proton inventory was performed in the pL (pH or pD) independent region of the profile (pL=8.0). A 0.1 M Na.EPPS buffer in ddH₂O was titrated to pH 8.0, with NaOH, and diluted in ddH₂O to 50 mM. A 0.1 M Na.EPPS was prepared in D₂O, and the buffer was titrated to pD 8.0 by adding 0.4 to the pH meter reading (Schowen and Schowen 1982)., using NaOD. This solution was diluted in D₂O to 50 mM.

Different volumes of both solutions were mixed according to Table 8, taking into consideration the slightly different molar volumes of H₂O and D₂O (Schowen and Schowen 1982).

TABLE 8
Volumes of D₂O and H₂O buffers used in the preparation
of the isotopic mixtures used in the proton inventory.

| Volume (μl) of H₂O | Volume (μl) of D₂O | Mole % |
|---|---|---|
| 50 mM Na.EPPS pH 8.0 | 50 mM Na.EPPS pD 8.0 | deuterium oxide |
| 800 | — | 0 |
| 700 | 100 | 12.38 |
| 600 | 200 | 24.77 |
| 500 | 300 | 37.15 |
| 400 | 400 | 49.53 |
| 300 | 500 | 61.92 |
| 200 | 600 | 74.30 |
| 100 | 700 | 86.68 |
| 0 | 800 | 100 |

For the rate constant measurements, Bipartite II DNAzyme (E1, SEQ. ID NO. 23) (final concentration 2.25 μM) was combined with trace amounts of S9 (SEQ. ID NO. 18), 5′ end labeled, and dried under vacuum. Reactions were ressuspended in the corresponding buffer (20 μl), and an aliquot of 2 μl was taken for time zero. To start the reaction, 2 μl of MgCl₂ (in D₂O) to a final concentration of 30 mM was added, so that the final concentration of buffer was 45 mM, and the final % mole deuterium oxide was increased (and taken into consideration in the proton inventory).

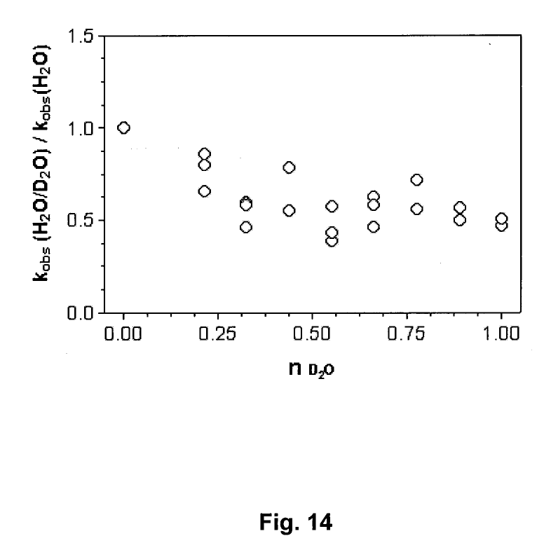

The reaction rate constant in isotopic mixtures of D₂O/H₂O was measured at the pL independent region of the profile (pL=8.0). The first-order rate constant k_{obs} (D₂O/H₂O) was normalized to the rate constant in H₂O (k_{obs} H₂O), and plotted against solvent isotopic composition, nD₂O. FIG. 14 shows the results. The first-order rate constants measured in varied solvent isotopic composition were normalized to the rate constant in H₂O, and plotted against molar ratio of D₂O (n D₂O).

The data obtained could fit a linear regression, but it could also fit a bulge-shaped curve (bulging down). A linear regression indicates that the isotope effect is generated by a single site. That means that one proton is transferred in the transition state (Schowen and Schowen 1982). A bulge-shaped curve could correspond to different models, one of which is that there two protons “in flight” in the transition state of the reaction (Schowen and Schowen 1982).

From the results shown in FIG. 14, it is not possible to clearly distinguish which model the data fits. However, the data indicates that there is at least one proton transferred in the transition state of Bipartite II catalyzed reaction.

### Example 15

Cross-Linking Experiments

In order to obtain information about the Bipartite II DNAzyme structure, a cross-linking experiment was designed. Five prime labeled Bipartite II DNAzyme (E1, SEQ. ID NO. 23) was incubated in the presence of high excess of an inactive substrate analog of S9 (SEQ. ID NO. 18), called dS9 (SEQ. ID NO. 86). Substrate dS9 (SEQ. ID NO. 86) has the sequence of substrate S9 (Table 3, SEQ. ID NO. 18) but it is entirely DNA. Upon irradiating the sample with a hand lamp at 254 nm, depending on the solution conditions used, a shift in the mobility of the enzyme band was observed in a 20% denaturing gel. That band was called the cross-linked species of Bipartite II DNAzyme (E1, SEQ. ID NO. 23).

For the cross-linking experiments, trace amounts of Bipartite II DNAzyme (E1, SEQ. ID NO. 23) (5′-end labeled) was combined with an inactive substrate analogue in high excess. Different conditions were used for each reaction as described below, and the samples were irradiated at 4° C. in a 40 μl volume, in an open 0.65 ml tube. A hand lamp able to irradiate at 254 nm was held on top of the tubes, always placed in the same height with respect to the solution (^{˜}2 cm from the solution). At the most, 12 samples were irradiated at a time, so that the distribution of light among different samples was about the same.

For cross-linking experiments in the presence of monovalent cations, 50 pmoles of Bipartite II DNAzyme (E1, SEQ. ID NO. 23) were 5′ end-labeled with P-32, purified in a 20% denaturing gel, and ressuspended in 20 μl of TE. 1 μl of Bipartite II DNAzyme (E1, SEQ. ID NO. 23) was combined with 50 pmoles dS9 (SEQ. ID NO. 86), and 8 μl H₂O, heat to 90° C. for 1 minute, and incubated at 23° C. for 5 minutes.

The reactions were lyophilized, and the pellets ressuspended in buffers containing 50 mM Na.HEPES pH 7.4, 20 mM EDTA, 3 M monovalent salt (NaCl, KCl or LiCl), 30 mM Co(NH₃)₆³⁺, or 10 mM MgCl₂; with a final volume of 40 μl. Samples were irradiated at 4° C. with an UV hand lamp, at 254 nm, during 30 minutes. Samples were ETOH precipitated, resuspended in denaturing dye, and cross-linked species analyzed in a 20% polyacrylamide gel.

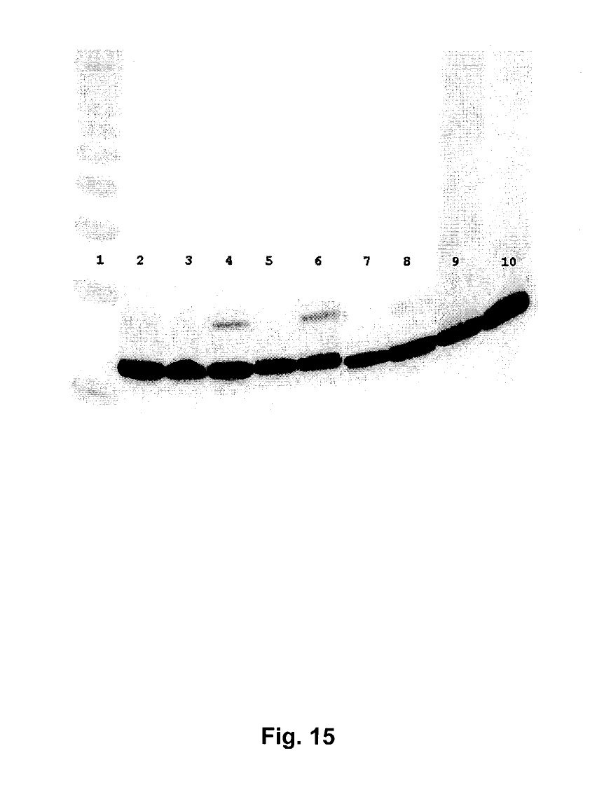

FIG. 15 shows the results obtained when using different solutions during irradiation of Bipartite II DNAzyme (E1, SEQ. ID NO. 23) and dS9 (SEQ. ID NO. 86). (Lanes: 1) 10 base pair ladder; 2) Bipartite II DNAzyme (E1, SEQ. ID NO. 23), dS9 (SEQ. ID NO. 86); 3) Bipartite II DNAzyme (E1, SEQ. ID NO. 23), 10 mM MgCl₂; 4) Bipartite II DNAzyme (E1, SEQ. ID NO. 23), dS9 (SEQ. ID NO. 86), 10 mM MgCl₂; 5) Bipartite II DNAzyme (E1, SEQ. ID NO. 23), 3M LiCl; 6) Bipartite II DNAzyme (E1, SEQ. ID NO. 23), dS9 (SEQ. ID NO. 86), 3M LiCl; 7) Bipartite II DNAzyme (E1, SEQ. ID NO. 23), 2M NaCl; 8) E1 (SEQ. ID NO. 23), dS9 (SEQ. ID NO. 86), 2M NaCl; 9) Bipartite II DNAzyme (E1, SEQ. ID NO. 23), 30 mM Co(NH₃)₆; 10) Bipartite II DNAzyme (E1, SEQ. ID NO. 23), dS9 (SEQ. ID NO. 86), 30 mM Co(NH₃)₆).

Results show that in the presence of substrate, and magnesium, Bipartite II DNAzyme (E1, SEQ. ID NO. 23) is forming a cross-linked structure within the catalytic motif of the DNAzyme. The cross-linked Bipartite II DNAzyme (E1, SEQ. ID NO. 23) is an intra-molecular cross-link formed within the Bipartite II DNAzyme (E1, SEQ. ID NO. 23) molecule. The cross-linked nucleotides reflect positions in the folded structure of the enzyme which are presumably close in space. Because the cross-link species of Bipartite II DNAzyme (E1, SEQ. ID NO. 23) only forms if substrate and certain divalent cations are present (such as magnesium), it is believed that the cross-linked Bipartite II DNAzyme (E1, SEQ. ID NO. 23) might represent an active form of the Bipartite II DNAzyme.

### Example 16

Synthesis and Design of Bipartite II DNAzymes that Target the Dpy-5 mRNA from C. Elegans

The potential of the Bipartite II DNAzyme to cleave a messenger RNA under physiological conditions was explored. Six different enzyme constructs targeting the dpy-5 mRNA from C. elegans, at different locations were designed, synthesized and tested for cleavage (Table 9, Seq. ID Nos. 32 to 45).

Target cleavage sites were designed based on the secondary structure of the RNA, in regions that did not participate in any duplex formation (target sites included bulges and loops). The DNA was synthesized using standard oligonucleotide synthesis techniques, in an ABI 392 DNA synthesizer, at Simon Fraser University. The glass beads had a fluorescein tag, so that resulting oligonucleotides had a 3′fluorescein (3′-6-FAM, CPG, Glen Research). To start the DNA synthesis, empty DNA synthesizing columns were rinsed with ammonium hydroxide, washed extensively with ddH₂O, dried, and packed with the fluorescein glass beads. The scale of DNA synthesis was 0.2 μmoles, and 4 mg of beads were used to pack each column. No special modification from standard DNA synthesis procedure was required with the fluorescein beads. Deprotection and cleavage from the beads involved overnight incubation of DNA in saturated ammonium hydroxide solutions at 55° C. When the oligonucleotides were completely deprotected, the solution color was green. Oligonucleotides were butanol precipitated and purified in a preparative polyacrylamide gel.

For each DNA enzyme construct designed, a defective mutant was synthesized as well. The oligonucleotides used as enzymes are shown in Table 3. To investigate whether the presence of a fluorescein at the 3′ end of the DNAzymes could affect cleavage, a version of Bipartite II DNAzyme (E1, SEQ. ID NO. 23) was designed and synthesized to carry a fluorescein at the 3′ end. A self-cleavage assay was performed in the presence of substrate S9 (SEQ. ID NO. 18) and resulted in full cleavage of substrate by the fluorescein labeled DNAzyme.

The presence of a fluorescein in Bipartite II DNAzyme (E1, SEQ. ID NO. 23) was investigated by scanning the DNA in a Cary UV-visible spectrophotometer, from 600 nm to 200 nm. Briefly, after synthesis and butanol precipitation, Bipartite II DNAzyme (E1, SEQ. ID NO. 23)-fluorescein was ressuspended in 200 μl TE. Five microliters were added to a 1 ml TE containing cuvet, and a peak at 495 mn indicated the presence of fluorescein.

The target dpy-5 RNA was in vitro transcribed from linear double-stranded plasmid, using a Promega ribo-probe in vitro transcription system, in the presence of α-P32-GTP, according to manufacturer's instructions. Following transcription the RNA was digested with DNAse (during 15 minutes at 37° C.), phenol: isoamyl extracted, chloroform extracted and ETOH precipitated in the presence of 6 μg of glycogen only. The RNA was purified in a sequencing size 5% denaturing polyacrylamide gel, and eluted in 0.5 M NH₄OAc, 10 mM Mg(OAc)₂, 1 mM Na.EDTA, and 0.1% SDS. For the cleavage assays with the different enzyme constructs, trace amounts of substrate were incubated with 8 pmoles of enzyme, in 50 mM Na.HEPES pH 7.4, 2 mM MgCl₂, 150 mM NaCl, 37° C., during 18 hours. Reaction aliquots were analyzed on an 8% denaturing polyacrylamide gel.

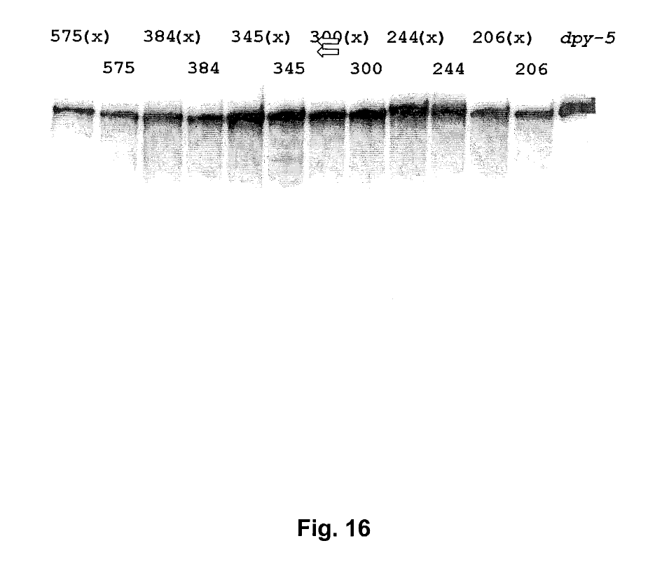

FIG. 16 is an electrophoresis gel depicting cleavage of the dpy-5 mRNA by different constructs of Bipartite II DNAzyme. Each lane represents a different reaction, containing a particular enzyme construct. Constructs containing (x) are the inactive mutants, because of a nucleotide substitution at the catalytic core. Arrows indicate cleaved fragments.

As expected, no cleavage by any enzyme was observed in the antisense RNA (data not shown). As shown in FIG. 16, the sense dpy-5 sense RNA (mRNA) was not cleaved by any inactive enzyme construct (X), but construct E345 (SEQ. ID NO. 38) was able to cleave it, under simulated physiological conditions (2 mM MgCl₂, 150 mM NaCl, 37° C., ^{˜}18 hours). This indicates that Bipartite II is able to identify its cleavage site in a long RNA substrate (such as the messenger RNA of dpy-5, which contains 855 bases). Table 9 shows the design of the constructs of Bipartite II DNAzymes used.

TABLE 9

Enzyme constructs designed to target the mRNA of dpy-5.

| | |
|---|---|
| Enzyme | 5′→3′ Sequence |

| | |
|---|---|
| E206 | CC AC GA AC AGGA GGTA GGGG TTCC GCTC GC TC AG CA |
| E206(X) | CC AC GA AC AGGA GGTA GGGG TTCC GCTT GC TC AG CA |
| E244 | AG CA TT GG AGGA GGTA GGGG TTCC GCTC GG AA CG CT |
| E244(X) | AG CA TT GG AGGA GGTA GGGG TTCC GCTT GG AA CG CT |
| E300 | GG CT CC TG AGGA GGTA GGGG TTCC GCTC CC TG GA GC |
| E300(X) | GG CT CC TG AGGA GGTA GGGG TTCC GCTT CC TG GA GC |
| E345 | TG GT CC GT AGGA GGTA GGGG TTCC GCTC CC GT CT GG |
| E345(X) | TG GT CC GT AGGA GGTA GGGG TTCC GCTT CC GT CT GG |
| E384 | GT CA TT TG AGGA GGTA GGGG TTCC GCTC GG AA TG TT |
| E384(X) | GT CA TT TG AGGA GGTA GGGG TTCC GCTT GG AA TG TT |
| E575 | GC AA GT CC AGGA GGTA GGGG TTCC GCTC GT CC GT CT |
| E575(X) | GC AA GT CC AGGA GGTA GGGG TTCC GCTT GT CC GT CT |
| E618 | TG GT TG TC AGGA GGTA GGGG TTCC GCTC CC GG TA AG |
| E618(X) | TG GT TG TC AGGA GGTA GGGG TTCC GCTT CC GG TA AG |

Bold regions correspond to the catalytic core.
The difference between the active and inactive construct (x) is a C to T mutation at the last nucleotide of the catalytic core. (Seq. ID Nos. 32 to 45).

As will be apparent to those skilled in the art, in the light of the foregoing disclosure, many alterations and modifications are possible in the practice of this invention without departing from the scope thereof. Accordingly, the scope of the invention is to be construed in accordance with the substance defined by the following claims.

### REFERENCES

Breaker, R. R. (1999). “In vitro selection of self-cleaving ribozymes and deoxyribozymes.” Intracellular ribozyme applications, Principles and Protocols, (Rossi, J. J. and Couture, L. A.), pp. 1, Horizon Scientific Press, Norfolk.

Breaker, R. R. and G. F. Joyce (1994). “A DNA enzyme that cleaves RNA.” Chemistry @ Biology 1: 223-229.

Breaker, R. R. and G. F. Joyce (1994b). “Inventing and improving ribozyme function: rational design versus iterative selection methods.” Tibtech 12: 268-275.

Breaker, R. R. and G. F. Joyce (1995). “A DNA enzyme with Mg2+-dependent RNA phosphoesterase activity.” Chemistry & Biology 2: 655-660.

Cleland, W. W., M. H. O'Leary, et al. (1977). “Isotope effects on enzyme-catalyzed reactions.” University Park Press, Baltimore.

Faulhammer, D. and M. Famulok (1996). “The Ca ion as a cofactor for a novel RNA-cleaving deoxyribozyme.” Angew. Chem. Int. Ed. Engl. 35: 2837-2841.

Faulhammer, D. and M. Famulok (1997). “Characterization and divalent metal-ion dependence of in vitro selected deoxiribozymes which cleave DNA/RNA chimeric oligonucleotides.” Journal of Molecular Biology 269: 188-202.

Fauzi, H., J. Kawakami, et al. (1997). “Analysis of the cleavage reaction of a trans-acting human hepatitis delta virus ribozyme.” Nucleic Acids Research 25: 3124-3130.

Fedor, M. J. and O. C. Uhlenbeck (1992). “Kinetics of intermolecular cleavage by the hammerhead ribozyme”. Biochemistry, 31:12042-12054.

Feldman, A. R. and D. Sen (2001). “A new and efficient DNA enzyme for the sequence specific cleavage of RNA”. Journal of Molecular Biology, 313:283-394.

Fersht A. (1999). Structure and mechanism in protein science: a guide to enzyme catalysis and protein folding. W. H. Freeman, New York.

Gesteland, R. F., T. R. Cech, et al. (1998). “The RNA world.” 2nd edition. Cold spring harbor Monograph series 37.

Geyer, R. C. and D. Sen (1997). “Evidence for the metal-cofactor independence of an RNA phosphodiester-cleaving DNA enzyme.” Chemistry & Biology 4: 579-593.

Geyer, R. C. and D. Sen (1998). “Lanthanide probes for a phosphodiester-cleaving lead-dependent, DNAzyme.” Journal of Molecular Biology 275: 483-489.

Guo, H. C. and R. A. Collins (1995). “Efficient trans-cleavage of a stem-loop RNA substrate by a ribozyme derived from Neurospora VS RNA.” EMBO J. 14: 368-376.

Hampel A. and Tritz R. (1989) RNA catalytic properties of the minimum (−)s TRSV sequence. Biochemistry 28, 4929-4933.

He, Q.-C., Zhou, J.-M., Zhou, D.-M., Nakamatsu, Y., Baba, T. and Taira, K. (2002). Comparison of metal-ion-dependent cleavage of RNA by DNA enzyme and hammerhead ribozyme. Biomacromolecules 3, 69-83.

Hegg, L. A. and M. J. Fedor (1995). “Kinetics and thermodynamics of intermolecular catalysis by hairpin ribozymes.” Biochemistry 34: 15813-15828.

Hertel, K. J., A. Pardi, et al. (1992). “Numbering system for the hammerhead.” Nucleic Acids Research 20(12): 3252.

Li, J., W. Zheng, et al. (2000). “In vitro selection and characterization of a highly efficient Zn(II)-dependent RNA cleaving deoxyribozyme.” Nucleic Acids Research 28(2): 481-488.

Lustig, B. and K. T. Jeang (2001). “Biological applications of hammerhead ribozymes as anti-viral molecules.” Curr. Med. Chem. 8: 1181-1187.

Muotri, A. R., L. V. Pereira, et al. (1999). “Ribozymes and anti-gene therapy: how a catalytic RNA can be used to inhibit gene function.” Gene 237: 303-310.

Murray, J. B., A. A. Seyhan, et al. (1998). “The hammerhead, hairpin and VS ribozymes are catalytically proficient in monovalent cations alone.” Chem. & Biol. 5: 587-595.

Nakano, S., D. M. Chadalavada, et al. (2000). “General acid-base catalysis in the mechanism of a hepatitis delta virus ribozyme.” Science 287: 1493-1497.

Nakano, S.-i. and P. C. Bevilacqua (2001). “Proton inventory of the genomic HDV ribozyme in Mg-containing solutions.” JACS 123: 11333-11334.

Peracchi, A. (2000). “Preferential activation of the 8-17 deoxyribozyme by Ca²⁺ ions. Evidence for the identity of 8-17 with the catalytic domain of the MG5 deoxyribozyme.” J. Biol. Chem. 275: 11693-11697.

Perrin D. M., Garestier T. and Helene C. (2001). Bridging the gap between proteins and nucleic acids: a metal independent RNAse A mimic with two protein-like functionalities. J. Am. Chem. Soc. 123, 1556-1563.

Rastogi, T. and R. A. Collins (1998). “Smaller, faster ribozymes reveal the catalytic core of Neurospora VS RNA.” Journal of Molecular Biology 277: 215-224.

Roth and R. D. Breaker (1998). “An amino acid as a cofactor for a catalytic polynucleotide.” Proc. Natl. Acad. Sci. USA 95: 6027-6031.

Santoro, S. S., G. F. Joyce, et al. (2000). “RNA cleavage by a DNA enzyme with extended chemical functionality.” J. Am. Chem. Soc. 122: 2433-2439.

Santoro, S. W. and G. F. Joyce (1997). “A general purpose RNA-cleaving DNA enzyme.” Proc. Natl. Acad. Sci. USA 94: 4262-4266.

Santoro, S. W. and G. F. Joyce (1998). “Mechanism and utility of an RNA-cleaving DNA enzyme.” Biochemistry 37(38): 13330-13342.

Saville, B. J. and R. A. Collins (1990). “A site-specific self-cleavage reaction performed by a novel RNA in Neurospora mitochondria.” Cell 61: 685-696.

Schowen, K. B. and R. L. Schowen (1982). “Solvent isotope effects in enzyme systems.” Methods in Enzymology 87: 551-606.

Shih I.-H. and Been M. D. (2000). Kinetic scheme for intermolecular RNA cleavage by a ribozyme derived from hepatitis delta virus RNA. Biochemistry 39, 9055-9066.

Stage-Zimmermann, T. K. and O. C. Uhlenbeck (1998). “Hammerhead ribozyme kinetics.” RNA 4: 875-889.

Sun, L. Q., M. J. Cairns, et al. (2000). “Catalytic nucleic acids: from lab to applications.” Pharmacol Rev 52: 325-347.

Symons, R. H. (1992). “Small catalytic RNAs.” Ann. Rev. Biochem. 61: 641-671.

Tuerk, C. and L. Gold (1990). “Systematic evolution of ligands by exponential enrichment: RNA ligands to bacteriophage T4 DNA polymerase.” Science 249: 505-510.

Wu, H.-N., Y.-L. Lin, et al. (1989). “Human hepatitis delta virus RNA subfragments contain an autocleavage activity.” Proc. Natl. Acad. Sci. USA 86: 1831-1835.

What is claimed is:

1. An RNA cleaving DNAzyme for cleaving a target RNA substrate, said DNAzyme comprising:
a. a catalytic core comprising SEQ. ID NO. 83; and
b. substrate binding arms proximate to the catalytic core.

2. The DNAzyme as claimed in claim 1, wherein the catalytic core is selected from the group consisting of SEQ. ID NO. 1, SEQ. ID NO. 2, SEQ. ID NO. 3, and SEQ. ID NO. 4.

3. The DNAzyme as claimed in claim 1, wherein the catalytic core comprises SEQ. ID NO. 1.

4. The DNAzyme as claimed in claim 1, wherein the catalytic core comprises SEQ. ID NO. 2.

5. The DNAzyme as claimed in claim 1, wherein the catalytic core comprises SEQ. ID NO. 3.

6. The DNAzyme as claimed in claim 1, wherein the catalytic core comprises SEQ. ID NO. 4.

7. The DNAzyme as claimed in claim 1, wherein the substrate binding arms flank the catalytic core.

8. The DNAzyme as claimed in claim 1, wherein the substrate binding arms are capable of Watson-Crick base pairing with the target RNA substrate.

9. The DNAzyme as claimed in claim 1, wherein the substrate binding arms comprise up to 7 nucleotides 5′ upstream of the catalytic core.

10. The DNAzyme as claimed in claim 1, wherein the substrate binding arms comprise more than 7 nucleotides 5′ upstream of the catalytic core

11. The DNAzyme as claimed in claim 1, wherein the substrate binding arms comprise up to 7 nucleotides 3′ downstream of the catalytic core.

12. The DNAzyme as claimed in claim 1, wherein the substrate binding arms comprise more than 7 nucleotides 3′ downstream of the catalytic core.

13. The DNAzyme as claimed in claim 9, wherein the substrate binding arms comprise between 5 and 8 nucleotides 3′ downstream of the catalytic core.

14. The DNAzyme as claimed in claim 1, wherein the DNAzyme cleaves the target RNA substrate at a sequence 5′-AANNN-3′, wherein N is any nucleotide.

15. The DNAzyme as claimed in claim 1, wherein the DNAzyme cleaves the target RNA substrate at a sequence 5′-AUNNN-3′, wherein N is any nucleotide.

16. The DNAzyme as claimed in claim 1, wherein the DNAzyme cleaves the target RNA substrate at a sequence 5′-ACNNN-3′, wherein N is any nucleotide.

17. The DNAzyme as claimed in claim 1 wherein the DNAzyme cleaves the target RNA substrate at a sequence 5′-AANN-3′, wherein N is any nucleotide.

18. A method of cleaving a target RNA molecule comprising cleaving the RNA molecule with the DNAzyme as claimed in claim 1.

19. The method as claimed in claim 18 wherein the target RNA molecule is derived from HIV.

20. The method as claimed in claim 18 wherein the target RNA molecule is derived from C. elegans.

21. The method as claimed in claim 18 wherein the target RNA molecule is cleaved inside a cell.

22. The method as claimed in claim 21, wherein the cell is infected with a virus.

23. The method as claimed in claim 22, wherein the virus is HIV.

24. The method as claimed in claim 22, wherein the target RNA molecule is a RNA molecule encoded by a viral gene.

25. The method as claimed in claim 24, wherein the viral gene is HIV-nef.

26. The method as claimed in claim 24, wherein the viral gene is HIV-rev.

27. The method as claimed in claim 24, wherein the viral gene is HIV-tat.

28. The method as claimed in claim 21, wherein the cell is infected with a microorganism.

29. The method as claimed in claim 28, wherein the target RNA molecule is a RNA molecule encoded by a microorganism gene.

30. The method as claimed in claim 21, wherein the target RNA molecule encodes for a disease causing gene product.

31. The method as claimed in claim 30, wherein the RNA molecule is encoded by a cancer causing gene.

32. The method as claimed in claim 18, wherein the RNA molecule is cleaved in vitro.

33. A self-cleaving DNAzyme having a catalytic core for cleaving an internal ribonucleotide, the catalytic core comprising SEQ. ID NO. 84.

34. The self-cleaving DNAzyme as claimed in claim 33, wherein the catalytic core is selected from the group consisting of SEQ. ID NO. 5, SEQ. ID NO. 6, SEQ. ID NO. 7, SEQ. ID NO. 8, SEQ. ID NO. 9, and SEQ. ID NO. 10.

35. The self-cleaving DNAzyme as claimed in claim 33, wherein the catalytic core comprises SEQ. ID NO. 5.

36. The self-cleaving DNAzyme as claimed in claim 33, wherein the catalytic core comprises SEQ. ID NO. 6.

37. The self-cleaving DNAzyme as claimed in claim 33, wherein the catalytic core comprises SEQ. ID NO. 7.

38. The self-cleaving DNAzyme as claimed in claim 32, wherein the catalytic core comprises SEQ. ID NO. 8

39. The self-cleaving DNAzyme as claimed in claim 32, wherein the catalytic core comprises SEQ. ID NO. 9

40. The self-cleaving DNAzyme as claimed in claim 32, wherein the catalytic core comprises SEQ.ID NO. 10.


SEQUENCE LISTING (86 sequences)

SEQ ID NO: 1 - DNA, 20 bp, Artificial Sequence
  Bipartite II DNAzyme catalytic core sequence
aggaggtaggggttccgctc

SEQ ID NO: 2 - DNA, 20 bp, Artificial Sequence
  Bipartite II DNAzyme alternate catalytic core sequence fromre-selection clones
tggaggtaggggttccgctc

SEQ ID NO: 3 - DNA, 20 bp, Artificial Sequence
  Bipartite II DNAzyme alternate catalytic core sequence fromre-selection clones
tcaaggtaggggttccgctc

SEQ ID NO: 4 - DNA, 20 bp, Artificial Sequence
  Bipartite II DNAzyme alternate catalytic core sequence fromre-selection clones
agaaggtaggggttccgctc

SEQ ID NO: 5 - DNA, 22 bp, Artificial Sequence
  Bipartite I DNAzyme catalytic core sequence
aggaggtaggggttccgctcca

SEQ ID NO: 6 - DNA, 22 bp, Artificial Sequence
  Bipartite I DNAzyme alternate catalytic core sequence fromselected clones
tcgaggtaggggttccgaacca

SEQ ID NO: 7 - DNA, 22 bp, Artificial Sequence
  Bipartite I DNAzyme alternate catalytic core sequence from selected clones
aggaggtaggggttccggacca

SEQ ID NO: 8 - DNA, 22 bp, Artificial Sequence
  Bipartite I DNAzyme alternate catalytic core sequence from selected clones
aggaggtaggggttccgatcca

SEQ ID NO: 9 - DNA, 22 bp, Artificial Sequence
  Bipartite I DNAzyme alternate catalytic core sequence from selected clones
aggaggtaggggttccgatcca

SEQ ID NO: 10 - DNA, 22 bp, Artificial Sequence
  Bipartite I DNAzyme alternate catalytic core sequence fromselected clones
acgaggtaggggttccgatcca

SEQ ID NO: 11 - DNA, 82 bp, Artificial Sequence
  Oligonucleotide sequence of library used for selection of Bipartite I DNAzyme
acgatagcagcagatgtcttacgnnnnnnnnnnnnnnnnnnnnnnnnnnnnnnnnnnnnnnnncgctcaatcggtaagtaac

SEQ ID NO: 12 - DNA, 25 bp, Artificial Sequence
  Primer rWT-bio
gacattgactttagcagccacttga

SEQ ID NO: 13 - DNA, 43 bp, Artificial Sequence
  Primer WT2
tgactttagcagccacttgaacgatagcagcagatgtcttacg

SEQ ID NO: 14 - DNA, 19 bp, Artificial Sequence
  Primer WT1
gttacttaccgattgagcg

SEQ ID NO: 15 - DNA, 30 bp, Artificial Sequence
  Primer 13RNA-bio
ttttggaauugaacgauagccgcagatgtc

SEQ ID NO: 16 - DNA, 12 bp, Artificial Sequence
  Primer WT-2short
gccgcagatgtc

SEQ ID NO: 17 - RNA, 26 nt, Artificial Sequence
  Substrate 7
ggaauugaacgauagccgcagauguc

SEQ ID NO: 18 - RNA, 20 nt, Artificial Sequence
  Substrate 9
ggaauugaacgauagccgca

SEQ ID NO: 19 - RNA, 19 nt, Artificial Sequence
  Substrate 31
ggaauugaacgauagccuu

SEQ ID NO: 20 - RNA, 21 nt, Human immunodeficiency virus
uuugcuauaagauggguggca

SEQ ID NO: 21 - RNA, 21 nt, Human immunodeficiency virus
agagcaagaaauggagccagu

SEQ ID NO: 22 - RNA, 22 nt, Human immunodeficiency virus
augagagugaaggagaaauauc

SEQ ID NO: 23 - DNA, 32 bp, Artificial Sequence
  Bipartite II DNAzyme E1
ggctaaggaggtaggggttccgctccaattcc

SEQ ID NO: 24 - DNA, 35 bp, Artificial Sequence
  Bipartite II DNAzyme E2
tgcggctaaggaggtaggggttccgctccaattcc

SEQ ID NO: 25 - DNA, 32 bp, Artificial Sequence
  Bipartite II DNAzyme E5
ggctaaggaggtaggggttccgctctaattcc

SEQ ID NO: 26 - DNA, 32 bp, Artificial Sequence
  Bipartite II DNAzyme E6
ggctaaggaggtaggggttccgctcaaattcc

SEQ ID NO: 27 - DNA, 32 bp, Artificial Sequence
  Bipartite II DNAzyme E7
ggctaaggaggtaggggttccgctcgaattcc

SEQ ID NO: 28 - DNA, 34 bp, Artificial Sequence
  Bipartite II DNAzyme E8
aaggctaaggaggtaggggttccgctccaattcc

SEQ ID NO: 29 - DNA, 36 bp, Artificial Sequence
  Bipartite II DNAzyme HIV-nef
tgccacccaggaggtaggggttccgctcatagcaaa

SEQ ID NO: 30 - DNA, 36 bp, Artificial Sequence
  Bipartite II DNAzyme HIV-tat
actggctcaggaggtaggggttccgctccttgctct

SEQ ID NO: 31 - DNA, 36 bp, Artificial Sequence
  Bipartite II DNAzyme HIV-env
gatatttcaggaggtaggggttccgctccactctca

SEQ ID NO: 32 - DNA, 36 bp, Artificial Sequence
  Bipartite II DNAzyme E206
ccacgaacaggaggtaggggttccgctcgctcagca

SEQ ID NO: 33 - DNA, 36 bp, Artificial Sequence
  Bipartite II DNAzyme E206(X)
ccacgaacaggaggtaggggttccgcttgctcagca

SEQ ID NO: 34 - DNA, 36 bp, Artificial Sequence
  Bipartite II DNAzyme E244
agcattggaggaggtaggggttccgctcggaacgct

SEQ ID NO: 35 - DNA, 36 bp, Artificial Sequence
  Bipartite II DNAzyme  E244(X)
agcattggaggaggtaggggttccgcttggaacgct

SEQ ID NO: 36 - DNA, 36 bp, Artificial Sequence
  Bipartite II DNAzyme E300
ggctcctgaggaggtaggggttccgctccctggagc

SEQ ID NO: 37 - DNA, 36 bp, Artificial Sequence
  Bipartite II DNAzyme E300(X)
ggctcctgaggaggtaggggttccgcttcctggagc

SEQ ID NO: 38 - DNA, 36 bp, Artificial Sequence
  Bipartite II DNAzyme E345
tggtccgtaggaggtaggggttccgctcccgtctgg

SEQ ID NO: 39 - DNA, 36 bp, Artificial Sequence
  Bipartite II DNAzyme E345(X)
tggtccgtaggaggtaggggttccgcttccgtctgg

SEQ ID NO: 40 - DNA, 36 bp, Artificial Sequence
  Bipartite II DNAzyme E384
gtcatttgaggaggtaggggttccgctcggaatgtt

SEQ ID NO: 41 - DNA, 36 bp, Artificial Sequence
  Bipartite II DNAzyme E384(X)
gtcatttgaggaggtaggggttccgcttggaatgtt

SEQ ID NO: 42 - DNA, 36 bp, Artificial Sequence
  Bipartite II DNAzyme E575
gcaagtccaggaggtaggggttccgctcgtccgtct

SEQ ID NO: 43 - DNA, 36 bp, Artificial Sequence
  Bipartite II DNAzyme E575(X)
gcaagtccaggaggtaggggttccgcttgtccgtct

SEQ ID NO: 44 - DNA, 36 bp, Artificial Sequence
  Bipartite II DNAzyme E618
tggttgtcaggaggtaggggttccgctcccggtaag

SEQ ID NO: 45 - DNA, 36 bp, Artificial Sequence
  Bipartite II DNAzyme E618(X)
tggttgtcaggaggtaggggttccgcttccggtaag

SEQ ID NO: 46 - DNA, 40 bp, Artificial Sequence
  Clone 6-61
tccaaagatcgaggtaggggttccgaaccaggtggcgtgc

SEQ ID NO: 47 - DNA, 37 bp, Artificial Sequence
  Clone 6-60
cgttgcctgaggaggtaggggttccggaccaattgtt

SEQ ID NO: 48 - DNA, 40 bp, Artificial Sequence
  Clone 6-63
gctcttaggaggtaggggttccgatccaggtggctgggta

SEQ ID NO: 49 - DNA, 42 bp, Artificial Sequence
  Clone 6-67
tctcgggcggcggaggaggtaggggttccgctccacaagggc

SEQ ID NO: 50 - DNA, 40 bp, Artificial Sequence
  Clone 12-17
tctctttctgcggaggaggtaggggttccgctccaagggc

SEQ ID NO: 51 - DNA, 40 bp, Artificial Sequence
  Clone 12-29
tctctttctgcagaggaggtaggggttccgctccaagggc

SEQ ID NO: 52 - DNA, 39 bp, Artificial Sequence
  Clone 12-6
ggcagcgaatagaggaggtaggggttccgctccaagggc

SEQ ID NO: 53 - DNA, 40 bp, Artificial Sequence; identical to SEQ ID NO: 48
  Clone 12-36

SEQ ID NO: 54 - DNA, 39 bp, Artificial Sequence
  Clone 12-8
gtgcttgcgacgaggtaggggttccgatccaatgggctg

SEQ ID NO: 55 - DNA, 74 bp, Artificial Sequence
  Bipartite I DNAzyme reselection Library A structure
ttttggaauugaacgauagccgcagatgtcnnnnnnnaggaggtaggggttccgctcnnnnnnncgctcaatcg

SEQ ID NO: 56 - DNA, 34 bp, Artificial Sequence
  Bipartite I re-selection clone sequence 1
caggctaaggaggtaggggttccgctccaattcc

SEQ ID NO: 57 - DNA, 34 bp, Artificial Sequence; identical to SEQ ID NO: 28
  Bipartite I re-selection clone sequence 2

SEQ ID NO: 58 - DNA, 34 bp, Artificial Sequence
  Bipartite I re-selection clone sequence 3
taggctaaggaggtaggggttccgctccaattcc

SEQ ID NO: 59 - DNA, 34 bp, Artificial Sequence
  Bipartite I re-selection clone sequence 4
aaggataaggaggtaggggttccgctccaattcc

SEQ ID NO: 60 - DNA, 33 bp, Artificial Sequence
  Bipartite I re-selection clone sequence 5
tggctaaggaggtaggggttccgctccaattcc

SEQ ID NO: 61 - DNA, 34 bp, Artificial Sequence
  Bipartite I re-selection clone sequence 6
gcggataaggaggtaggggttccgctccaattcc

SEQ ID NO: 62 - DNA, 34 bp, Artificial Sequence
  Bipartite I re-selection clone sequence 7
gaggctaaggaggtaggggttccgctccaattcc

SEQ ID NO: 63 - DNA, 34 bp, Artificial Sequence; identical to SEQ ID NO: 56
  Bipartite I re-selection clone sequence 8

SEQ ID NO: 64 - DNA, 34 bp, Artificial Sequence
  Bipartite I re-selection clone sequence 9
gcagctatggaggtaggggttccgctccaattcc

SEQ ID NO: 65 - DNA, 34 bp, Artificial Sequence
  Bipartite I re-selection clone sequence 10
caggctatcaaggtaggggttccgctccaattcc

SEQ ID NO: 66 - DNA, 34 bp, Artificial Sequence
  Bipartite I re-selection clone sequence 11
gaggctaagaaggtaggggttccgctccaattcc

SEQ ID NO: 67 - DNA, 74 bp, Artificial Sequence
  Bipartite I DNAzyme Clone 12-17 Full Sequence
ttttggaauugaacgauagccgcagatgtccaggctaaggaggtaggggttccgctccaattcccgctcaatcg

SEQ ID NO: 68 - RNA, 26 nt, Artificial Sequence
  Bipartite II test substrate 1
ggaauugaacgauagccgcagauguc

SEQ ID NO: 69 - RNA, 26 nt, Artificial Sequence
  Bipartite II test substrate 2
ggaauugaacgbuagccgcagauguc

SEQ ID NO: 70 - RNA, 26 nt, Artificial Sequence
  Bipartite II test substrate 3
ggaauugaachauagccgcagauguc

SEQ ID NO: 71 - RNA, 26 nt, Artificial Sequence
  Bipartite II test substrate 4
ggaauugaadgauagccgcagauguc

SEQ ID NO: 72 - RNA, 26 nt, Artificial Sequence
  Bipartite II test substrate 5
ggaauugaccgauagccgcagauguc

SEQ ID NO: 73 - RNA, 26 nt, Artificial Sequence
  Bipartite II test substrate 6
ggaauugaucgauagccgcagauguc

SEQ ID NO: 74 - RNA, 26 nt, Artificial Sequence
  Bipartite II test substrate 7
ggaauugagcgauagccgcagauguc

SEQ ID NO: 75 - RNA, 26 nt, Artificial Sequence
  Bipartite II test substrate 8
ggaauuggacgauagccgcagauguc

SEQ ID NO: 76 - RNA, 26 nt, Artificial Sequence
  Bipartite II test substrate 9
ggaauugcacgauagccgcagauguc

SEQ ID NO: 77 - RNA, 26 nt, Artificial Sequence
  Bipartite II test substrate 10
ggaauuguacgauagccgcagauguc

SEQ ID NO: 78 - RNA, 26 nt, Artificial Sequence
  Bipartite II test substrate 11
ggaauucaacgauagccgcagauguc

SEQ ID NO: 79 - RNA, 26 nt, Artificial Sequence
  Bipartite II test substrate 12
ggaauuaaacgauagccgcagauguc

SEQ ID NO: 80 - RNA, 26 nt, Artificial Sequence
  Bipartite II test substrate 13
ggaauuuaacgauagccgcagauguc

SEQ ID NO: 81 - RNA, 25 nt, Artificial Sequence
  Bipartite II test substrate 14
ggaauugaacguagccgcagauguc

SEQ ID NO: 82 - RNA, 23 nt, Artificial Sequence
  Bipartite II test substrate 15
ggaauugaauagccgcagauguc

SEQ ID NO: 83 - DNA, 20 bp, Artificial Sequence
  Bipartite II DNAzyme catalytic core consensus sequence
nnnaggtaggggttccgctc

SEQ ID NO: 84 - DNA, 22 bp, Artificial Sequence
  Bipartite I DNAzyme general catalytic core sequence
nngaggtaggggttccgnncca

SEQ ID NO: 85 - DNA, 44 bp, Artificial Sequence
  DNA template for transcription of Substrate 7.
gacatctgcggctatcgttcaattccctatagtgagtcgtatta

SEQ ID NO: 86 - DNA, 20 bp, Artificial Sequence
  Substrate dS9
ggaattgaacgatagccgca